(19) United States
(12) Patent Application Publication
(10) Pub. No.: US 2023/0045112 A1
(43) Pub. Date: Feb. 9, 2023

(54) DISULFIRAM AND OTHER REDOX-RELATED COMPOSITIONS FOR BRAIN TUMORS

(71) Applicant: Texas Tech University System, Lubbock, TX (US)

(72) Inventors: Kalkunte S. Srivenugopal, Amarillo, TX (US); Ameya M. Paranjpe, Amarillo, TX (US)

(21) Appl. No.: 17/956,249

(22) Filed: Sep. 29, 2022

Related U.S. Application Data
(63) Continuation-in-part of application No. 15/116,757, filed on Aug. 4, 2016.
(60) Provisional application No. 61/936,455, filed on Feb. 6, 2014.

(51) Int. Cl. A61K 33/34; A61K 31/194; A61K 47/02; A61K 9/48; A61K 31/404

(52) U.S. Cl. CPC A61K 33/34; A61K 31/194; A61K 47/02; A61K 9/4833; A61K 31/404

(57) ABSTRACT

The present disclosure provides a compositions and methods of inhibiting O⁶-methylguanine DNA methyltransferase in human tumor cells by providing an effective amount of an agent that directly or indirectly inhibits the O⁶-methylguanine DNA methyltransferase in a pharmaceutically acceptable carrier, wherein the amount is effective to potentiate an anti-tumor activity of one or more alkylating agents, platinum drugs, or antimetabolites, wherein tumor cells are triggered into programmed cell death.

### CROSS-REFERENCE TO RELATED APPLICATIONS

This application is a continuation in part of U.S. Non Provisional application Ser. No. 15/116,757, filed on Aug. 4, 2016 which claims priority to U.S. National Phase of International Application No. PCT/US2015/014789, filed on Feb. 6, 2015, which claims the benefit under 35 U.S.C. § 119(e) of U.S. Provisional Application No. 61/936,455, filed Feb. 6, 2014. All of which are hereby incorporated by reference in their entirety.

### STATEMENT OF FEDERALLY FUNDED RESEARCH

This invention was made with government support under Grant No RO3CA125872 awarded by the National Institutes of Health (NIH). The government has certain rights in the invention.

### TECHNICAL FIELD OF THE INVENTION

The present invention relates in general to the field of methods of treating cancer, specifically to disulfiram compositions and other redox-perturbing compounds and methods of making and using analogues for the treatment of cancer.

### BACKGROUND OF THE INVENTION

Without limiting the scope of the invention, its background is described in connection with methods of treating brain cancer, as an example.

The incidence of brain tumors in adults and pediatric patients has seen a continued increase in recent years with an estimated 17,000 new cases diagnosed and 12,000 deaths every year in the United States. Brain tumors are also the second leading cause of cancer deaths among pediatric patients. Gliomas, medulloblastomas, and other brain cancers remain the most therapeutically challenging and chemotherapy using hydrophobic alkylating agents that cross the blood-brain barrier is a mainstay in their treatment. The clinically used alkylating agents also sensitize the brain tumors to radiation and therefore, there is a great need for strengthening/improving the efficacy of the monofunctional and bifunctional alkylating drugs used for central nervous system cancers. Disulfiram (DSF) has been shown to increase the cytotoxicity of many anticancer drugs such as the cisplatin, gemcitabine, paclitaxel and 5-fluorouracil.

O⁶-methylguanine DNA methyltransferase (MGMT) is a DNA repair protein expressed variably in human tissues and functions to protect the genome against mutations from the alkylating agents of endogenous and environmental origins. In up to 80% of brain cancers, it is highly expressed and its repair function interferes with the cytotoxic actions of the alkylating agents (nitrosoureas, temozolomide etc) used in the chemotherapy. This is because, MGMT scavenges the alkyl groups introduced into the DNA by the drugs and nullifies the tumor cell killing. There are very few inhibitors for MGMT, and the one in clinical trials causes prolonged inhibition of DNA repair, and this has not been useful for successful therapy as explained in the narrative above. Disulfiram is expected to overcome this problem. An inhibitor of MGMT called 06-benzylguanine (BG) went into clinical trials about 10 years ago, to inhibit all of MGMT in tumors and then give the alkylating drugs to patients to introduce more damage to the DNA, and thus eliminate the tumor cells in the brain. However, BG suppresses the MGMT activity in both normal and tumor tissues for a long time, and this suppression in the bone marrow stem cells causes greater damage in them, leading to deficient production of blood cells, and discontinuance of therapy.

U.S. Pat. No. 6,548,540, entitled, “Method of treating cancer using dithiocarbamate derivatives,” discloses dithiocarbamate, particularly tetraethylthiuram disulfide, and thiocarbamate anions strongly inhibit the growth of cancer cells of a variety of cell types. Such inhibitory effect is enhanced by heavy metal ions such as copper ions, cytokines and ceruloplasmin and a method is presented for using tetraethylthiuram disulfide to reduce tumor growth, and to potentiate the effect of other anticancer agents.

U.S. Pat. No. 6,288,110, entitled, “Pharmaceutical compositions comprising disulfiram,” discloses disulfiram (tetraethylthiuram disulfide) to inhibit angiogenesis and to be useful in the treatment of angiogenesis-dependent disorders, including neoplasms, and to prevent cell hyperproliferation and formation of clots along or around medical devices.

### SUMMARY OF THE INVENTION

As embodied and broadly described herein, an aspect of the present disclosure relates to a pharmaceutical composition comprising at least one of: cupric diethyldithiocarbamate (Cu-DDC), a DDC pro-drug, KSS-72, or 2,4-Dimethylene glutaric acid (DMG) in a pharmaceutically acceptable carrier for use in the treatment of tumor cells wherein the Cu-DDC, DDC pro-drug, KSS-72, or DMG directly or indirectly inhibit O⁶-methylguanine DNA methyltransferase in the tumor cells, wherein the amount is effective to potentiate an anti-tumor activity of one or more alkylating agents, platinum drugs, or antimetabolites, wherein tumor cells are triggered into programmed cell death. In one aspect, the Cu-DDC, DDC pro-drug, KSS-72, or DMG further comprise an albumin encapsulation, a liposomal encapsulation, or both. In another aspect, the DDC pro-drug is administered in combination with a metal chelate that includes an ion selected from the group consisting of arsenic, bismuth, cobalt, copper, chromium, gallium, gold iron, manganese, nickel, silver, titanium, vanadium, selenium and zinc. In another aspect, the pharmaceutical composition further comprises a boronic acid hybrid, an ethacynic acid linked DDC, an N-acetyl lysine-linked DDC, or a gamma-glutamyl p-amido benzyl DDC. In another aspect, the DMG is provided in an amount sufficient to inhibit alpha keto glutarate (alpha KG) dehydrogenase to increase oxidative stress that inhibits O⁶-methylguanine DNA methyltransferase and triggers programmed cell death. In another aspect, the pharmaceutical composition is administered in a dosage of between 10, 20, 30 40, 50, 50, 60, 70, 75, 80, 90, 100, 200, 250, 300, 400, 500, 600, 700, 750, 800, 900 or 1000 mg of the Cu-DDC, DDC pro-drug, KSS-72, or DMG per day of body weight. In another aspect, the tumor cells are human brain tumor cells and the composition is formulated for intravenous or in situ administration.

The pharmaceutical composition of claim 1, wherein the tumor cells are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease. In another aspect, the one or more alkylating agents, platinum drugs, or antimetabolites are selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a,-9-hexohydrofuro(3′,:6,7)naphtho(2,3- d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna. In another aspect, the Cu-DDC, DDC pro-drug, KSS-72, or DMG composition is provided in an amount that inhibits O⁶-methylguanine DNA methyltransferase (MGMT) in situ. In another aspect, the pharmaceutical composition further comprises temozolomide. In another aspect, the pharmaceutical composition further comprises an activatable Cu-DDC, KSS-72, or DMG, wherein the Cu-DDC, KSS-72, or DMG are activatable in vivo with near infrared radiation, or the activatable Cu-DDC, DDC pro-drug, KSS-72, or DMG are conjugated with glutathione.

As embodied and broadly described herein, an aspect of the present disclosure relates to a pharmaceutical composition comprising an effective amount of an NAD(P)H Quinone Dehydrogenase 1 (NQO1) substrate in a pharmaceutically acceptable carrier that generates reactive oxygen species (ROS) in tumor cells, wherein processing of the NQO1 substrate in situ triggers programmed cell death in tumor cells by inhibiting O⁶-methylguanine DNA methyltransferase. In one aspect, the pharmaceutical composition is formulated for oral, parenterally, intravenous, or in situ administration. In another aspect, the pharmaceutical composition is administered in a dosage of between about 10, 20, 30 40, 50, 50, 60, 70, 75, 80, 90, 100, 200, 250, 300, 400, 500, 600, 700, 750, 800, 900 or 1000 mg per day of body weight. In another aspect, the tumor cells are human brain tumor cells. In another aspect, the tumor cells are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease. In another aspect, the pharmaceutical composition further comprises a cytotoxic agents selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13 (9H,15H) dione, lurtotecan, 7- [2-(N-isopropylamino)ethyl]-(20 S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a, -9-hexohydrofuro(3′,′:6, 7)naphtho(2,3 d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna. In another aspect, the Cu-DDC, KSS-72, or DMG composition is provided in an amount to inhibit O⁶-methylguanine DNA methyltransferase (MGMT) in situ. In another aspect, the pharmaceutical composition further comprises temozolomide. In another aspect, the pharmaceutical composition further comprises an activatable Cu-DDC, KSS-72, or DMG, wherein the Cu-DDC, KSS-72, or DMG are activatable in vivo with near infrared radiation, or the activatable Cu-DDC, KSS-72, or DMG is conjugated with glutathione.

As embodied and broadly described herein, an aspect of the present disclosure relates to a pharmaceutical composition comprising an effective amount of cupric diethyldithiocarbamate [Cu-DDC], KSS-72, or 2,4-Dimethylene glutaric acid (DMG) in a pharmaceutically acceptable carrier to inhibit 06-methylguanine DNA methyltransferase in tumor cells, wherein the Cu-DDC is formulated for oral, parenterally or in situ administration.

As embodied and broadly described herein, an aspect of the present disclosure relates to a chemotherapy composition comprising cupric diethyldithiocarbamate [Cu-DDC], KSS-72, or 2,4-Dimethylene glutaric acid (DMG), or activatable pro-GNQ, or pro-NQ-IND, disposed in a pharmaceutically acceptable carrier.

As embodied and broadly described herein, an aspect of the present disclosure relates to a chemotherapy composition comprising an effective amount of cupric diethyldithiocarbamate [Cu-DDC], Cu-DDC pro-drug, KSS-72, 2,4-Dimethylene glutaric acid (DMG), GNQ, pro-GNQ, NQ-IND, in an amount sufficient to inhibit an O⁶-methylguanine DNA methyltransferase disposed in a pharmaceutically acceptable carrier.

As embodied and broadly described herein, an aspect of the present disclosure relates to a theranostic agent comprising an NAD(P)H Quinone Dehydrogenase 1 (NQO1) substrate that generates reactive oxygen species (ROS) in tumor cells in cellulo and that comprises a detectable agent, wherein the detectable agent is used to diagnose the presence or absence of tumor cells, and when cleaved, the NQO1 substrate reduces or eliminates the tumor cells. In one aspect, the detectable agent is a fluorophore. In another aspect, the NQO1 substrate is GNQ or a functionalized derivative with substitutions selected from halogen, aryl, alkyl, or alkoxy group substitutions. In another aspect, the NQO1 substrate is inducible. In another aspect, the theranostic agent further comprises one or more one or more alkylating agents, platinum drugs, or antimetabolites are selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a, -9-hexohydrofuro(3′,′:6,7)naphtho(2,3- d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna. In another aspect, the theranostic agent further comprises temozolomide. In another aspect, the NQO1 substrate is:

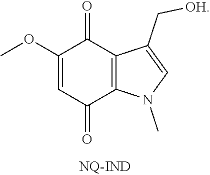 [SMILES: COC1=CC(=O)c2c(c(CO)cn2C)C1=O]

### DESCRIPTION OF THE DRAWINGS

For a more complete understanding of the features and advantages of the present invention, reference is now made to the detailed description of the invention along with the accompanying FIGS. and in which:

FIGS. 1A-1D show concentration-dependent inhibition of MGMT activity and MGMT degradation induced by DSF and its derivatives in human brain tumor cells. FIG. 1A shows inhibition of the DNA repair activity of purified recombinant MGMT (rMGMT) and cellular MGMT by increasing DSF concentrations. rMGMT or UW228 cell-free extracts were first treated with DSF at concentrations shown for 20 min at room temperature followed by the addition of DNA substrate. FIG. 1B shows inhibition of MGMT activity and loss of MGMT protein in T98G and UW228 cells. FIG. 1C shows the effect of CuDSF on MGMT activity and protein levels in UW228 and T98G cells. FIG. 1D shows the effect of copper and DSF treatments on MGMT.

FIGS. 2A-2C show time-dependent elimination of MGMT protein in brain tumor cells by DSF, CuDSF and Cu+DSF. FIG. 2A shows T98G and UW228 cells were treated 50 μM DSF. At times indicated, MGMT activity and protein levels were assessed. FIG. 2B shows potent inactivation of MGMT by 10 μM CuDSF in T98G and UW228 cells. MGMT Protein levels in drug-treated UW228 cells are shown. FIG. 2C shows inhibition of MGMT in tumor cells after a combined treatment of 0.1 μM CuCl2 and DSF.

FIG. 3A shows immunofluorescence decreased MGMT protein levels in DSF-treated UW228 cells. Drug treatments and exposure times are shown in labels. FIG. 3B shows depletion and repletion kinetics of MGMT protein following BG and DSF treatments. FIG. 3C shows that Cys145 of MGMT is the target site for DSF. FIG. 3D shows DSF abrogates the binding of the BG probe to cellular MGMT. UW228 cell extracts proficient in MGMT were incubated with DSF or N-ethylmaleimide (0.5 mM) for 20 min followed by BG-PEG-biotin as described for the purified proteins.

FIGS. 4A-4E show proteasomal degradation of DSF modified MGMT. FIG. 4A shows in vitro degradation of DSF treated rMGMT protein. FIG. 4B shows the proteasome inhibitor PS-341 curtails the degradation of DSF-modified MGMT protein in brain tumor cells. FIG. 4C shows DSF increases the DNA damage induced by MGMT-targeted alkylating agents. FIG. 4D shows G2/M blockade induced by BCNU is enhanced and extended in the presence of DSF. Histograms showing results of FACS-based cell cycle analysis of UW228 cells treated with BCNU alone, DSF alone, and BCNU+DSF combination. Asynchronous cultures were treated or untreated with DSF (50 μM for 12 h) and then incubated with 100 μM BCNU. The cells were trypsinized at times indicated and analyzed by flow cytometry. FIG. 4E shows enhanced levels of apoptosis markers—cleaved caspase 3 and cleaved PARP after DSF+BCNU treatments compared with BCNU alone.

FIGS. 5A-5G show DSF preexposure sensitizes the brain tumor cells to the clinically used alkylating agents. FIG. 5A shows cell survival after DSF treatment alone. UW228 cells were treated with increasing concentrations of DSF (1-100 μM) for 24 hours. FIG. 5B shows TMZ mediated cell killing with and without DSF preexposure in UW228 cells. FIG. 5C shows BCNU mediated cell killing with and without DSF preexposure in UW228 cells. FIG. 5D shows DSF did not increase the cytotoxicity of melphalan, an N7, but not an 06-alkylator of guanine in UW228 cells. FIG. 5E shows DSF did not potentiate the TMZ-induced cytotoxicity in the MGMT-deficient U87 malignant glioma cells. FIG. 5F shows DSF did not potentiate BCNU-induced cell killing in the MGMT-deficient U87 cells. In all these cases, the tumor cells were treated or untreated with 50 μM DSF for 12 hours followed by the alkylating drugs for 4 days before the MTT assays. FIG. 5G shows soft agar colony formation assay for cell survival analysis.

FIGS. 6A-6C show significant downregulation of MGMT activity and protein in mouse brain and liver tissues after DSF (150 mg/kg) administration. FIG. 6A shows DSF treatment resulted in a sustained 40% inhibition of brain MGMT activity starting at 9 hours (upper panel). Western blot analysis (bottom panel) shows a gradual decrease of MGMT protein in the same time course. FIG. 6B shows decreased hepatic MGMT activity (60% inhibition, upper panel) and protein levels (lower panel) after DSF administration. FIG. 6C shows the consequences of MGMT inhibition by DSF and increased in tumor sensitivity to alkylating agents.

FIG. 7 shows disulfiram, decomposition, and Cu-DDC formation. DSF is a symmetrical compound, is reduced and split in the middle to yield two dithiocarbamate (DDC) groups in the GI tract or blood. If copper ions are available, a stable coordinate complex Cu-DDC is formed. The physical appearance of Cu-DDC as fine black particles is shown on the left. If Cu is not around, part of the DDC undergoes methylation (active component) forming adducts with active site cysteines of enzymes. NPL4 is a protein important in the processing of ubiquitinated proteins was shown to be a direct target of Cu-DDC, the resulting proteostatic effects are listed (Skrott, et al., “Alcohol-abuse drug disulfiram targets cancer via p97 segregase adaptor NPL4”, Nature 2017 Dec. 14; 552(7684):194-199, 552, pages 194-199 (2017)).

FIGS. 8A to 8C show, elevated expression of NQO1 in human cancers (FIG. 8A); absence of NQO1 in human astrocytes and its abundance in brain cancer cell lines (FIG. 8B); IHC staining of GBM-12 tumor- and 3-lapachone to cytotoxic agents either through DNA alkylation or production of ROS through futile redox cycling (FIG. 8C).

FIGS. 9A and 9B shows the selective generation of peroxides but not superoxides by Cu-DDC in SF-188.

FIGS. 10A and 10B show that, while Cu-DDC upregulated the wt p53, it dramatically eliminated the mutant p53 found in UW228 medulloblastoma and other GBMs.

FIG. 11 shows that irrespective of the p53 status, Cu-DDC triggered dramatic cell death as detected by Annexin-V assay.

FIG. 12 shows that BSA nanoparticles (NPs) loaded with Cu-DDC showed significant antitumor efficacy against the DAOY medulloblastoma orthotopic xenografts.

FIGS. 13A to 13C show that cancer cells including the SNB GBM cells show a dramatic increase in cytotoxicity when the DDC drugs are combined with copper gluconate.

FIG. 14 shows the synthetic pathway for the various compounds of the present invention.

FIGS. 15A to 15D show: cytotoxicity of GNQ against different brain tumor cells.

FIG. 16 shows the synthetic pathway for GNQ.

FIGS. 17A to 17D show that GNQ acts as an NQO1 substrate and generates Reactive oxygen species (ROS).

FIGS. 18A to 18G show: FIG. 18A, 18B show complete suppression of intracranial DAOY medulloblastoma growth by GNQ. GNQ was given IP at 25 mg/kg/d, 5 d/wk for about 4 weeks. (FIG. 18C) No changes in body weight as a surrogate marker for toxicity were noted. (FIG. 18D) Ex-vivo bioluminescence from the isolated brains. (FIG. 18E) Accumulation of GNQ in the brain when compared to plasma. (FIG. 18F) H&E staining of mouse brain sections showing the elimination of GBM by GNQ. (FIG. 18G) Mean tumor volume radiance in the GNQ-treated group was compared with that in the control.

FIG. 19 shows the infrared NQO1 fluorophore NIR-ASM (prepared by linking dicyanoisophorone with the NQO1 substrate quinone propionic acid), which was injected at one mg/kg intravenously to the nude mice bearing NQO1-proficient AS549 tumors on the left flank and NQO1-deficient MDAMB-231 tumors on the right flank. The animals were imaged in a IVIS imager at 500-640 nm. As can be seen, only the NQO1-positive A549 tumors showed fluorescent signals within 5 min, and the NQO1-null tumors did not show any after 3 hours, demonstrating the specificity of the imaging reagent.

FIGS. 20A to 20E show: FIG. 20A, 20B show the structures ES936, NQ-IND. FIG. 20C shows the effect of NQ-IND and ES936 on the NQO1 activity. FIG. 20D shows the cytotoxicity of NQ-IND against cancer and normal cell lines. FIG. 20E shows the intracellular ROS levels induced by NQ-IND.

FIG. 21A to 21D show the inhibition of MGMT activity (FIG. 21A) and its degradation (FIG. 21B) induced by GNQ in DAOY cells. (FIG. 21C) Immunofluorescence shows the decreased MGMT protein levels in GNQ treated cells. (FIG. 21D) GNQ potentiates the cytotoxicity of TMZ.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the abbreviation DSF denotes 1,1′,1″,1′″-[disulfanediylbis(carbonothioylnitrilo)]tetraethane called disulfiram which is an FDA approved drug used as alcohol abuse deterrent based on the inhibitory activity on aldehyde dehydrogenase having the structure:

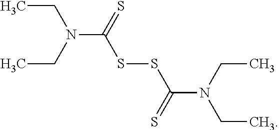 [SMILES: CCN(CC)C(=S)SSC(=S)N(CC)CC]

It undergoes thiol-disulfide exchange, also known as S-thiolation, targeting specific protein sulfhydryl groups and the critical cysteine residues in proteins. If another cysteine exists in the vicinity, intramolecular S—S bonds can result.

As used herein the abbreviation CuDSF denotes copper chelated disulfiram.

As used herein the abbreviation BG denotes O⁶-benzylguanine.

As used herein the abbreviation BCNU denotes 1,3-bis-2-chloroethylnitrosourea.

As used herein the abbreviation TMZ denotes temozolomide.

As used herein the abbreviation MGMT denotes O⁶-methylguanine DNA methyltransferase.

As used herein the abbreviation PS341 denotes bortezomib.

As used herein the abbreviation rMGMT denotes recombinant MGMT protein.

As used herein the abbreviation ALDH denotes aldehyde dehydrogenase.

As used herein the abbreviation ub denotes ubiquitin.

As used herein the abbreviation PARP denotes poly ADP-ribose polymerase.

As used herein the abbreviation Cu-DDC denotes cupric diethyldithiocarbamate, and Cu-DDC pro-drug refers to a precursor that upon processing becomes a Cu-DDC.

As used herein the abbreviation DMG denotes 2,4-Dimethylene glutaric acid.

As used herein the abbreviation GNQ is used interchangeably with GNQ-9 and denotes a molecule with the structure:

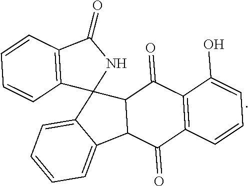 [SMILES: O=C1NC2(c3ccccc31)c1ccccc1C1C(=O)c3cccc(O)c3C(=O)C12]

As used herein the abbreviation NQ-IND denotes a molecule with the structure:

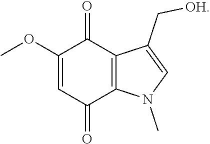 [SMILES: COC1=CC(=O)c2c(c(CO)cn2C)C1=O]

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, a “pharmaceutical salt” includes, but is not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like.

As used herein, the term “treatment” denotes an intervention performed with the intention of preventing the development or altering the pathology or symptoms of a disorder and refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

As used herein the term “dosing” and “treatment” denotes any process, action, application, therapy or the like, wherein a subject, particularly a human being, is rendered medical aid with the object of improving the subject's condition, either directly or indirectly.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human); and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “cytotoxic agent” denotes compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

As used herein, the term “theranostic” refers to agents that can function as both a diagnostic and a therapeutic agent. The theranostics of the present invention are useful in the individualized and comprehensive treatment strategies comprising direct treatment and elimination or reduction of tumors and the planning and the assessment of tumor responses. For example, the doses for targeted reactive oxygen species (ROS) therapy can employ treatment doses derived from the imaging data.

The present invention fulfills an urgent need for inhibiting the DNA repair protein MGMT (O6-methylguanine-DNA methyltransferase) and improving the efficacy of alkylating agents. MGMT is highly expressed in human cancers and prevents the formation of cytotoxic lesions in alkylated DNA. Current clinical trials involving MGMT depletion by O⁶-benzylguanine (BG), although promising, are beset with severe toxicity to the bone marrow, which has necessitated the transduction of BG-resistant MGMT gene into hematopoietic stem cells.

The present disclosure provides disulfiram as a direct and mechanism-based inhibitor of the human DNA repair protein MGMT. The present disclosure provides a composition that curtails MGMT for a shorter time and still be able incur sufficient damage in the tumor DNA. The present disclosure provides disulfiram, a FDA-approved drug for treatment of alcoholism, is an excellent inhibitor of MGMT.

Disulfiram binds with a critical cysteine residue at the active site of MGMT and turns off DNA repair. Disulfiram has the ability to penetrate the blood brain barrier, and we have shown that it strongly inhibits MGMT in brain tumor cells and mouse brain. The failure of chemotherapy and not being able to improve the quality of life in pediatric and adult patients with brain tumors is largely due to the overexpression of MGMT. Disulfiram is very safe (in people not drinking alcohol), does not induce any toxicity and can be given in large amounts orally to enable sustained availability to the brain. The pharmacology, dose, adverse effects and safety issues for disulfiram have been very well worked out.

There are very few inhibitors for MGMT, and the one in clinical trials caused prolonged inhibition of DNA repair, which has not been useful for successful therapy. Disulfiram as disclosed herein overcomes this problem.

The present disclosure provides disulfiram compositions to affect human MGMT, which removes the mutagenic O⁶-alkyl groups from guanines, and thus renders the normal tissues and brain tumors resistant to alkylation DNA damage. The present disclosure provides DSF, copper-chelated DSF (CuDSF) or CuCl2-DSF combination as treatments to inhibit the MGMT activity in two brain tumor cell lines in a rapid and dose dependent manner. The drug treatments resulted in the loss of MGMT protein from tumor cells and the degradation occurred through the ubiquitin-proteasome pathway. Evidence showed that Cys145, a reactive cysteine, critical for DNA repair was the sole site of DSF modification in the MGMT protein. DSF was a weaker inhibitor of MGMT, compared to the established O⁶-benzylguanine, nevertheless, the 24-36 hours suppression of MGMT activity in cell cultures vastly increased the alkylation-induced DNA interstrand crosslinking, G2/M cell cycle blockade, cytotoxicity and the levels of apoptotic markers. Mice treated with DSF showed significantly attenuated levels of MGMT activity and protein in the liver and brain tissues. The present disclosure provides strong and direct inhibition of MGMT-mediated DNA repair by the nontoxic DSF and support the repurposing of this brain penetrating drug for glioma therapy. Cysteine 145 of MGMT accepts the alkyl groups for pharmacological intervention. Cys145 has a pKa of 4.8 due to its microenvironment and is susceptible for glutathionylation and nitrosylation. S-Thiolation and S-nitrosylation are reversible posttranslational mechanisms that gauge the intracellular redox and transduce them into functional responses.

The present disclosure provides two nontoxic small molecules which readily react with reactive cysteines, namely, the NCX-4016 (nitro-aspirin capable of S-nitrosylation) and disulfiram (capable of thiol-conjugation).

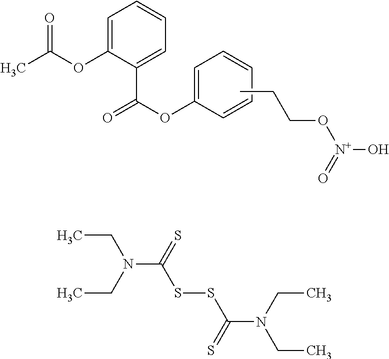

NCX-4016, also called a fatty aspirin, is degraded by plasma and tissue esterases to release NO in a sustained manner. In three MGMT-proficient human cancer cell lines (HT29, T47D, and HCT116), nitro-aspirin at very low concentrations (5-10 μM) caused a 90% inhibition of MGMT activity within 1 hour of exposure. Interestingly, the MGMT protein also disappeared with similar kinetics after NCX-4016 treatment; approx. 80-90% of MGMT was degraded after 5 μM NO-aspirin treatment for 2 hours. These data are highly comparable or better than those reported for BG. Further, purified MGMT or tumor cell extracts exposed to NCX-4016 failed to bind the biotin-labeled BG, indicating Cys145 to be the site of nitrosylation. More than 60% of MGMT protein was regenerated at 24 hours when NO-aspirin treated HT29 cells were post-incubated in drug-free medium, indicating a transient inhibition and rapid repletion. Disulfiram (DS), the alcohol deterrent drug, also curtailed MGMT activity in HT29 and HCT116 cells with a 20 hour 400 μM treatment causing a 95% inhibition. Disulfiram at 200 μM induced about 70% degradation of MGMT at 12 hours. Other redox-sensitive proteins such as the wild-type and mutant p53, NF-κB, and ubiquitin E1 were all degraded by disulfiram in a dose-dependent manner. In mice a single injection (100 mg/kg) of NCX-4016 causes 50-70% inhibition of MGMT activity in mice brain and liver. Because NO-aspirin, is non-toxic (IC50>500 μM for cell lines), yields a chemopreventive by-product, unlikely to elicit tumor resistance, and is lipophilic enough to cross the blood brain barrier (BBB), as such it can be well exploited for glioma therapy. The redox-regulated proteins are ‘druggable’ and highlight options for redox-driven therapeutic strategies.

O⁶-Methylguanine DNA-methyltransferase (MGMT) is a unique antimutagenic DNA repair protein that plays a crucial role in the defense against alkylating agents, particularly, those that generate the O⁶-alkylguanines. Guanine is the most preferred base for alkylation, and the adducts at the O⁶-guanine are particularly critical, because, the O⁶-alkylguanines pair aberrantly with thymine, resulting in GC to AT transitions. MGMT repairs O⁶-alkylguanine and O⁴-alkylthymine lesions by transferring the alkyl groups to an active site cysteine residue (Cys145) in the protein in a stoichiometric and suicidal reaction, so that the guanine in the DNA is simply restored in an error-free direct reversal reaction. Because, the alkyl group is covalently bound to the protein, MGMT is functionally inactivated after each reaction, and the inactive protein is degraded through the ubiquitin proteolytic pathway. MGMT is abundantly expressed in liver and other normal tissues, but is present at very low levels in the bone marrow and normal brain. The repair function of MGMT is essential for the removal of O⁶-guanine alkylations introduced by the carcinogens present in cooked meat, endogenous metabolites such as the S-adenosylmethionine, nitrosated amino acids and tobacco smoke, and maintaining genomic stability. MGMT appears to have a strong linkage with another public health problem, namely, the chronic alcohol abuse and the resulting pathological effects in liver and brain as well. A number of studies have described the suppression of MGMT and an increased alkylation damage following acute or chronic alcohol intake. Disulfiram (DSF, bis-diethylthiocarbamoyl disulfide), also known as Antabuse, is a carbamate derivative clinically used for treating alcoholism and more recently for cocaine addiction. DSF is a relatively nontoxic substance when administered alone, but markedly alters the metabolism of alcohol by irreversibly inhibiting the hepatic aldehyde dehydrogenase (ALDH) and causing an accumulation of acetaldehyde and consequent aversion to further drinking. DSF and its metabolites form mixed disulfide bridges with a critical cysteine (Cys302) near the active site region of ALDH to inactivate the enzyme. Recently, the inventors showed that DSF reacts similarly with a number of redox-sensitive proteins such as the p53 tumor suppressor, NF-κB, and ub-activating enzyme E1 and lead to their degradation. MGMT is highly expressed in about 80% of brain tumors and other cancers. Paradoxically, its antimutagenic function interferes with the cytotoxic actions of anticancer alkylating agents. This is because MGMT effectively repairs the O⁶-methylguanine and O⁶-chloroethylguanine lesions induced by methylating agents (temozolomide, dacarbazine and procarbazine) and chloroethylating agents (BCNU and CCNU) respectively, thereby preventing the generation of mutagenic lesions and interstrand DNA crosslinks. Consequently, MGMT has emerged as a central determinant of tumor resistance to alkylating agents. In view of this therapeutic relevance, MGMT has been extensively targeted for inhibitor development. Much success has been achieved through the design of psuedosubstrate inhibitors, namely, the O⁶-benzylguanine (BG) and O⁶-[4-bromothenyl] guanine (Patrin-2), which are currently undergoing clinical trials. In this biochemical strategy, the free base inhibitors (BG) are first administered to inhibit MGMT and create a DNA repair-deficient state followed by alkylating agents to increase the DNA damage and antitumor efficacy. BG is a specific and powerful inhibitor of MGMT and causes a prolonged suppression of DNA repair (48-72 hours) in cultured tumor cells. While this approach has shown a positive outcome in cultured cells and xenograft settings, a significant drawback is the excess of bone marrow toxicity encountered in patients enrolled in BG+alkylating agent combination regimens. Hematopoietic stem cells contain very low levels of MGMT, whose inactivation by BG predisposes them to excessive alkylation damage, which results in therapy discontinuance and necessitates the use of alkylating drugs at sub-therapeutic levels. This problem has prompted a gene therapy approach involving the transduction of BG-resistant MGMT genes (G156A or P140K) in to the hematopoietic stem cells. However, the cost, complexity, and safety issues make this approach cumbersome and impractical. The considerations above justify an urgent need for new and transient inhibitors for human MGMT. To design better and rational inhibitors for MGMT, the reactivity of Cys145 was exploited, which accepts the alkyl residues in the self-inactivating reaction. This active site cysteine has a low pKa of 4.5, and reacts readily with glutathione forming a mixed disulphide linkage. Cysteine 145 of MGMT is also a good substrate for nitrosylation. Therefore, the inventors hypothesized that drugs/small molecules with strong affinity for reactive cysteines will be able bind and inactivate the MGMT protein and disulfiram shows as such a candidate. The present disclosure characterizes MGMT inhibition by DSF, the augmented alkylation damage and a synergistic cytotoxicity and shows an increased carcinogenic risk in alcoholic patients taking disulfiram has also been discussed.

CuDSF was synthesized by mixing equimolar amounts of DSF and CuCl2 for 24 hours followed by extraction with chloroform as described previously. The final product was dried and stored in a desiccator. Hexa-histidine tagged human MGMT was expressed in E. coli and purified as described previously.

The DNA repair activity of MGMT was measured by the transfer of [3H]-labeled methyl groups from the O⁶-position of guanine in the DNA substrate to the MGMT protein as described previously. The DNA substrate enriched in O6-methylguanine was prepared by reacting [3H]-methylnitrosourea (GE Healthcare, 60 Ci/mmol). Briefly, the cell pellets were washed with cold PBS, disrupted by sonication in the assay buffer (30 mM Tris-HCl pH 7.5, 0.5 mM DTT, 0.5 mM EDTA, 5% glycerol, and 20 μM spermidine) and centrifuged. The extracts (50-150 μg protein) were supplemented with the [3H]-DNA (10,000 cpm) and incubated at 37° C. The reactions were terminated by the addition of TCA, the DNA substrate was hydrolyzed at 80° C., and following filtration on glass fiber discs; the radioactivity present in protein precipitates was quantitated.

After trypsinization, the cell pellets were washed with cold PBS, and subjected to sonication in 50 mM Tris-HCl (pH 8.0) containing 1% glycerol, 1 mM EDTA, 0.5 mM PMSF and 2 mM benzamidine and centrifuged. Equal protein amounts from different treatments were electrophoresed on 12% SDS-polyacrylamide gels. Proteins were electro-transferred to Immobilon-P membranes. The membranes were blocked with 5% non-fat dry milk in Trisbuffered saline (TBS; pH 8.0) containing 0.1% Tween 20 for 3 hours, and subsequently incubated with appropriate primary antibodies. Antigen-antibody complexes were visualized by enhanced chemiluminiscence (Pierce Company). Band intensities were quantified using a Bio-Rad Versa Doc Imaging system.

UW228 cells were cultured on sterile coverslips and treated with DSF for 12 hours. The treated and untreated cells were fixed with 4% paraformaldehyde for 20 minutes and washed with PBS. They were blocked with 3% BSA containing 0.2% Triton X-100 for 3 hours. Cells were incubated with the anti-MGMT antibody overnight at 4° C., washed thrice with PBS and incubated with Alexa Fluor-488 goat anti-mouse IgG (Invitrogen) for 1 hour. Cells were counterstained with Hoechst to observe the nuclei, washed and mounted on slides. Images were acquired and quantitated using an Olympus IX 81 fluorescence microscope.

Proteolysis assays in the presence of rabbit reticulocyte lysates (30 μl final vol.) were performed in Tris-HCl (30 mM; pH 8.0) containing 0.5 mM DTT, 4 mM MgCl2, 1 mM ATP, 1 μg ubiquitin, 0.7 μg rMGMT or 0.7 μg DSF treated rMGMT. DSF-exposed MGMT samples were dialyzed to remove the residual drug prior to the degradation assays. The reactions were initiated with the addition of 10 μl rabbit reticulocyte lysate (Promega), incubated at 37° C. for 15-40 minutes, electrophoresed and immunoblotted using anti-MGMT antibodies. The protein bands were quantified by densitometry.

Sixty percent confluent cells were treated with 50 μM DSF for 12 hours. This was followed by 100 μM BCNU. Untreated cells and cells treated with DSF and BCNU alone were used as controls. After the treatments, cells were allowed to grow for 24 and 48 hours. At each of these time points cells were harvested and fixed in 70% ethanol. The cells were then washed with PBS and re-suspended in the presence of RNase (1 μg/ml) and propidium iodide (PI, 50 μg/ml) for 30 minutes. Cell cycle histograms were generated using a BD Accuri C6 flow cytometer.

UW228 cells pre-treated with 50 μM DSF for 12 hours were treated with melphalan (0-48 hours, 100 NM) or BCNU (0-72 hours, 100NM). DNA from cells was isolated by lysis in TE buffer (10 mM Tris, 1 mM EDTA, pH 8.0) containing 0.5% SDS, and 100 μg/mlRNase A and proteinase K (1 mg/ml) digestion. DNA was precipitated with ethanol and dissolved in TE buffer. The drug-induced DNA crosslinking was measured by the ethidium bromide fluorescence assay as described previously. Briefly, 5-10 μg DNA was dissolved in assay buffer (20 mM potassium phosphate and 2 mM EDTA, pH 11.8) in duplicate. One set of tubes was heated at 100° C. for 10 min and cooled to room temperature. Ethidium bromide was added to 1 μg/ml, and the fluorescence was measured (305 nm excitation and 585 nm emission) using an LS-50 variable wavelength spectrofluorometer (Perkin Elmer). The fluorescence readings were used to compute the crosslink index, CLI, as described earlier.

These were performed using the yellow tetrazolium dye (3-(4, 5-dimethylthiazolyl-2)-2, 5-diphenyl tetrazolium bromide) (MTT) as described previously. Cells (15,000/well) in 24- well plates were treated with DSF (50 μM) for 12 hours before exposure to the alkylating agents. The purple color developed due to formazan was measured at 570 nm using a SPECTRAFluor Plus plate reader (Tecan Inc.). Cell proliferation was also assessed by colony formation assays on soft agar. In these assays, UW228 cells treated with 50 μM DSF were suspended in media containing DMEM, 20% FBS, 0.4% agarose, non-essential amino acids and BCNU (0-75 μM) or TMZ (0-750 Ten thousand cells from these suspensions were layered on 3 ml of the same medium containing 0.6% agar in 60 mm culture plates. Seven to ten days after plating, cell colonies in triplicate plates were counted and cell survival computed.

Mice weighing 19-21 g were administered a single dose of 150 mg/kg DSF dissolved in 50 μl DMSO i.p. injections (6 animals/group). Control mice received DMSO alone. The animals were sacrificed at 1, 3, 6, 9 and 12 hours after drug administration. The liver and brain tissues were harvested, washed, and homogenized in a buffer (0.5% Triton-X, 50 mM Tris-HCl buffer (pH 8.0) containing 3% glycerol, 50 mM NaCl, 1 mM EDTA, 0.5 mM PMSF and 2 mM benzamidine) using a Polytron. The lysates were centrifuged at 12,000 g and the resulting extracts were used for MGMT activity and western blot analyses.

The western blotting and immunocytochemical analyses were performed four times independent of each other. Results were assessed by Student's t test. Significance was defined as P<0.05. Power analysis was used to calculate the number of animals required to achieve a statistical power of >80%.

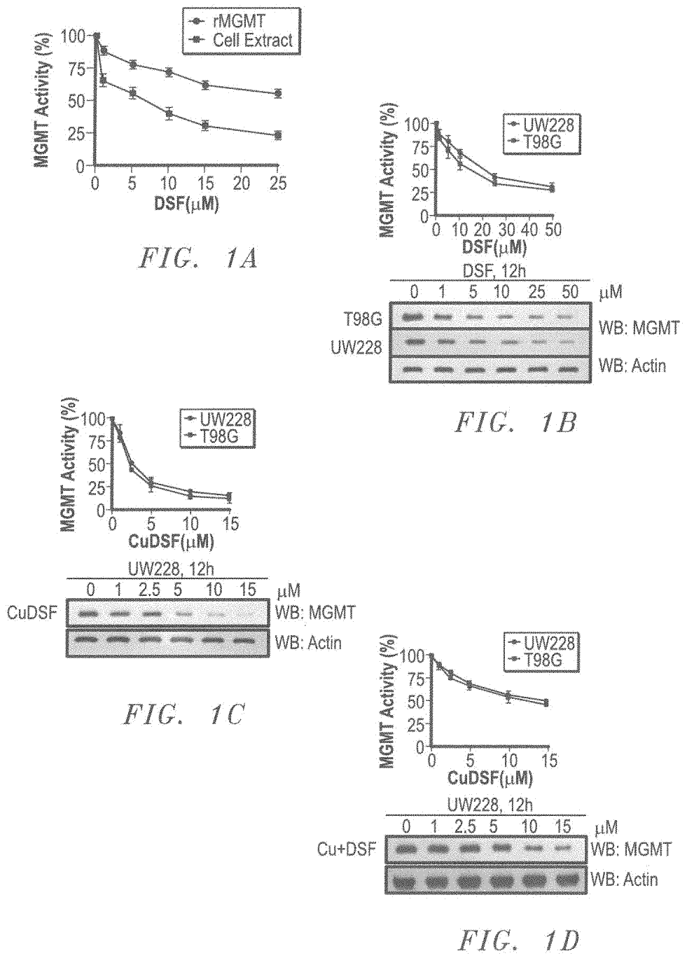

FIGS. 1A-1D show concentration-dependent inhibition of MGMT activity and MGMT degradation induced by DSF and its derivatives in human brain tumor cells. FIG. 1A shows inhibition of the DNA repair activity of purified recombinant MGMT (rMGMT) and cellular MGMT by increasing DSF concentrations. rMGMT or UW228 cell-free extracts were first treated with DSF at concentrations shown for 20 minutes at room temperature followed by the addition of DNA substrate. FIG. 1B shows inhibition of MGMT activity and loss of MGMT protein in T98G and UW228 cells. Tumor cell monolayers were exposed to DSF at concentrations specified for 12 hours. Cells were trypsinized, washed and MGMT activity was determined in cell extracts (top panel). The same extracts were western blotted for MGMT protein. WB, western blot. FIG. 1C shows the effect of CuDSF on MGMT activity and protein levels in UW228 and T98G cells. Cells were exposed to CuDSF and analyzed for MGMT activity and protein as above. FIG. 1D shows the effect of copper and DSF treatments on MGMT. Cells were incubated with 0.1 μM CuCl2 and DSF at concentrations shown for 12 hours before determining the MGMT activity and protein levels. The initial studies involved the treatment of the recombinant MGMT protein (rMGMT) and UW228 cell-free extracts (which contained MGMT) with DSF and quantitation the DNA repair activity. While 25 μM DSF inhibited the purified MGMT by 40%, the repair activity in the extracts was curtailed by 75%, suggesting a direct effect of the compound on MGMT protein (FIG. 1A). Next, the MGMT-proficient human brain tumor cell lines, T98G and UW228 were treated with DSF (0-500 μM) for 12 hours and the MGMT activity was measured in the extracts therefrom. In both cell lines, MGMT activity was inhibited by DSF in a dose-dependent fashion with approx. 70% inhibition in cells exposed to 50 μM DSF. Western blot analysis showed a gradual loss of MGMT protein, consistent with the extent of inhibition after 12 hours DSF-treatment (FIG. 1B). DSF binds copper with a great affinity, and the CuDSF was more potent in this property with 10-15 μM drug eliminating >90% of the MGMT activity and protein in UW228 cells after 12 hours treatment (FIG. 1C). The UW228 cells were exposed to 0.1 μM CuCl2 in combination with DSF; this schedule, however, resulted in modest inhibition of MGMT activity and protein levels (FIG. 1D). These results confirm that DSF and its metabolites are capable of inactivating the MGMT protein and lead to its breakdown in human cancer cells. To delineate the appropriate time for alkylating drug treatments following MGMT inhibition, next, the time course of MGMT depletion by these three interventions was studied.

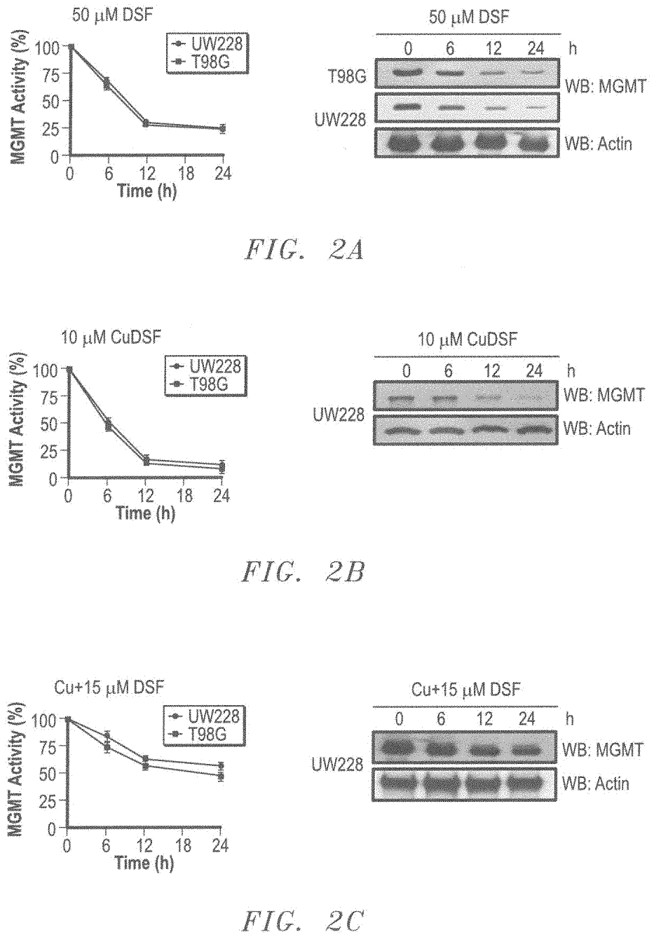

FIGS. 2A-2C show time-dependent elimination of MGMT protein in brain tumor cells by DSF, CuDSF and Cu+DSF. FIG. 2A shows T98G and UW228 cells were treated 50 μM DSF. At times indicated, MGMT activity and protein levels were assessed. FIG. 2B shows potent inactivation of MGMT by 10 μM CuDSF in T98G and UW228 cells. MGMT Protein levels in drug-treated UW228 cells are shown. FIG. 2C shows Inhibition of MGMT in tumor cells after a combined treatment of 0.1 μM CuCl2 and DSF. The presence of 50 μM DSF in the culture medium caused a maximal 75% inhibition of MGMT activity and protein at 12 hours, which was maintained till 24 hours (FIG. 2A). In comparison, CuDSF, at a 5-fold lesser concentration (10 μM) than DSF, elicited a >90% inhibition of MGMT activity and protein during the same period (FIG. 2B). Consistent with the results from FIG. 1D, the combination of Cu and DSF was less potent, with only 40-50% inhibition (FIG. 2C). Collectively, the findings in FIGS. 1 and 2 reveal that DSF is capable of rapidly inactivating the MGMT and maintain the inhibition to allow the induction of increased alkylation damage.

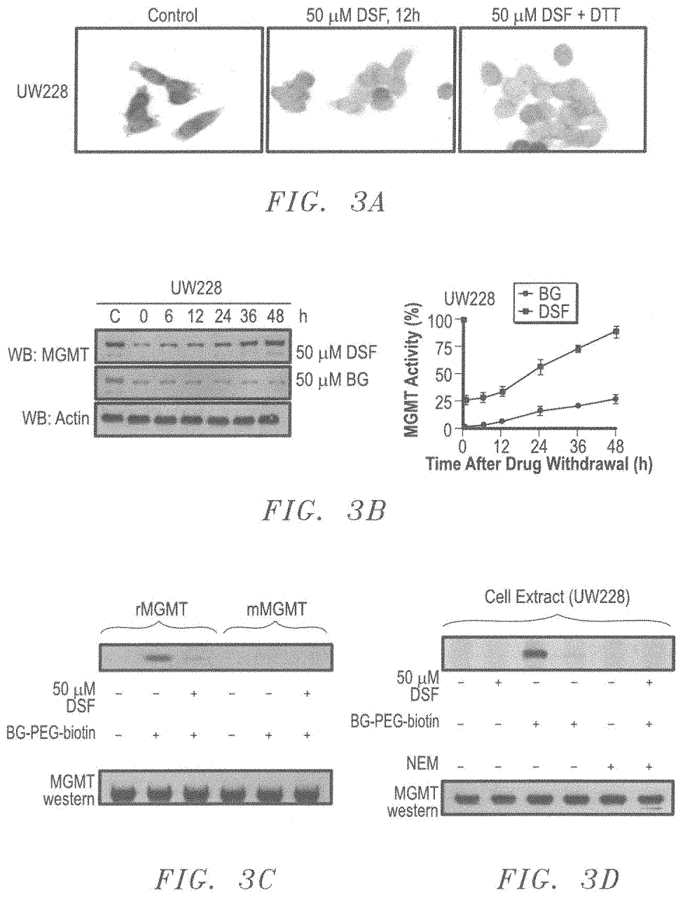

FIG. 3A shows immunoflourecence decreased MGMT protein levels in DSF-treated UW228 cells. Drug treatments and exposure times are shown in labels. In the last panel, DSF-treated cells were exposed to 10 mM DTT for 20 min before the antibody treatment. FIG. 3B shows depletion and repletion kinetics of MGMT protein following BG and DSF treatments. UW228 cells were exposed to 50 μM DSF or 50 μM BG for 12 hours followed by washing and suspension of cells in drug-free media. Cells were trypsinized at times shown. Levels of MGMT activity and protein were determined in cell extracts. Evidence that DSF blocks the active site cysteine (cys145) in human MGMT protein. FIG. 3C shows evidence that Cys145 of MGMT is the target site for DSF. Wild type rMGMT and mutant rMGMT (C145A) proteins were incubated with 50 μM DSF in 40 mM Tris-HCl, pH 8.0, 1 mM EDTA for 20 minutes at 37° C. BG-PEG-biotin (5 μM) was then added and incubations continued for 15 minutes. The samples were electrophoresed, blotted and probed with Streptavidin-HRP to detect the protein-bound biotin (upper panel). The blot was reprobed with antibodies to MGMT (lower panel). FIG. 3D shows DSF abrogates the binding of the BG probe to cellular MGMT. UW228 cell extracts proficient in MGMT were incubated with DSF or N-ethylmaleimide (0.5 mM) for 20 minutes followed by BG-PEG-biotin as described for the purified proteins. Streptavidin-HRP was used to probe the resulting blot (upper panel). The blot reprobed with MGMT antibody shows equal protein loading (lower panel).

To confirm the data obtained by western blotting in FIGS. 1A-D and 2A-C, immunostaining of MGMT in UW228 cells treated with DSF was performed. The representative photomicrographs in FIG. 3A shows that the antibody-specific staining for MGMT was markedly less in DSF-treated cells. To test whether the thiol linkages introduced by DSF on the protein are reversible, in some experiments, the DSF-treated cells were exposed to 10 mM DTT for 30 minutes prior to treatment with MGMT antibodies. The intensity of antibody staining did not change after DTT, indicating the non-reversibility of the modification. Further, DTT was unable to reverse the inhibition of MGMT activity by DSF (not shown). Thus, unlike the mixed disulfides introduced in S-thiolation reactions, the disulfiram linkages introduced in the MGMT protein appear largely irreversible.

MGMT performs a stoichiometric reaction to accomplish the DNA repair and is not recycled to its active form. Instead, the inactivated protein is degraded and fresh translation has to occur to restore the MGMT levels in cells. The rate and extent of this recovery is an important factor in the extent of damage introduced to the normal cell genomes such as the bone marrow stem cells. Therefore, the MGMT depletion and repletion kinetics after DSF treatment was compared with that by BG, an established inhibitor of MGMT, currently in clinical trials. In these experiments, the UW228 cells were incubated with 50 μM DSF or 50 μM BG for 12 hours, and then, the cultures were washed to remove the residual drugs followed by resuspension in drug-free media to allow MGMT regeneration. MGMT protein and activity levels were analyzed during the depletion and repletion (FIG. 3B). While BG resulted in 100% loss of MGMT activity, DSF produced a 75% inhibition at 12 hours post-drug treatments (FIG. 3B, right panel). However, the post-recovery of MGMT activity was very slow in BG-treated cultures compared to that in DSF treatment. While the MGMT activity recovered to 25% of control levels at 48 hours post-BG, it reached ˜95% in case of post-DSF at the same interval (FIG. 3B, right panel). MGMT protein levels as determined by western analyses during the depletion/repletion cycle were consistent with and fully reflected the DNA repair activity profiles (FIG. 3B, left panel). These data suggest that DSF functions as a transient inhibitor of MGMT, and that DSF inactivated MGMT may undergo an accelerated proteolysis compared to the benzylated MGMT protein. Since the longer MGMT suppression is likely to mediate a continued accumulation of alkylation DNA damage, our observations suggest that in contrast to BG, the DSF-induced short term inhibition of MGMT is likely to be beneficial in rescuing the host tissues from continued genomic injury. The human MGMT protein possesses 5 cysteine residues, of which the Cys145, which accepts the alkyl groups, is the most reactive. In the first approach, to test whether DSF reacts with cys145, a biotin-labeled BG probe was used, which binds specifically to this cysteine residue. Purified rMGMT protein was incubated with BG-PEG-biotin before or after treatments with DSF, followed by SDS-PAGE and detection of the protein-bound biotin using the streptavidin-HRP on the blots. When the protein was exposed to DSF followed by BG-PEGbiotin, there was a significant reduction in binding of the probe (FIG. 3C, lanes 1-3). Further, the purified mutant MGMT protein was used in which the Cys145 has been replaced with alanine in this assay. The BG-biotin probe failed to bind this mutant protein with or without DSF treatment (FIG. 3C, lanes 4-6). When the entire blot was reprobed with MGMT antibody, the rMGMT used in the assays was detected at equal levels (FIG. 3C, lower panel). Similarly, when the MGMT containing cell extracts were treated or untreated with DSF prior to labeling with the BG-PEG-biotin probe, DSF eliminated the western blot signal (FIG. 3D). Nethylmalamide (NEM) used as a control to block the active site cys145 of MGMT, also prevented the BG-labeling of the protein (FIG. 3D). These data clearly demonstrate that DSF modifies the active site cys145 of MGMT, which in turn, inactivates the DNA repair function.

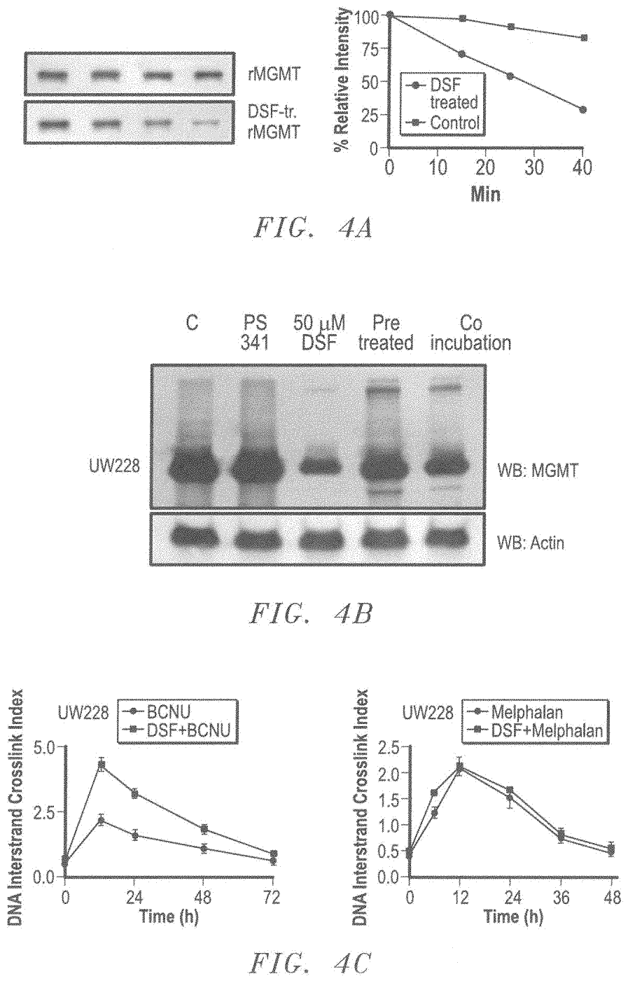

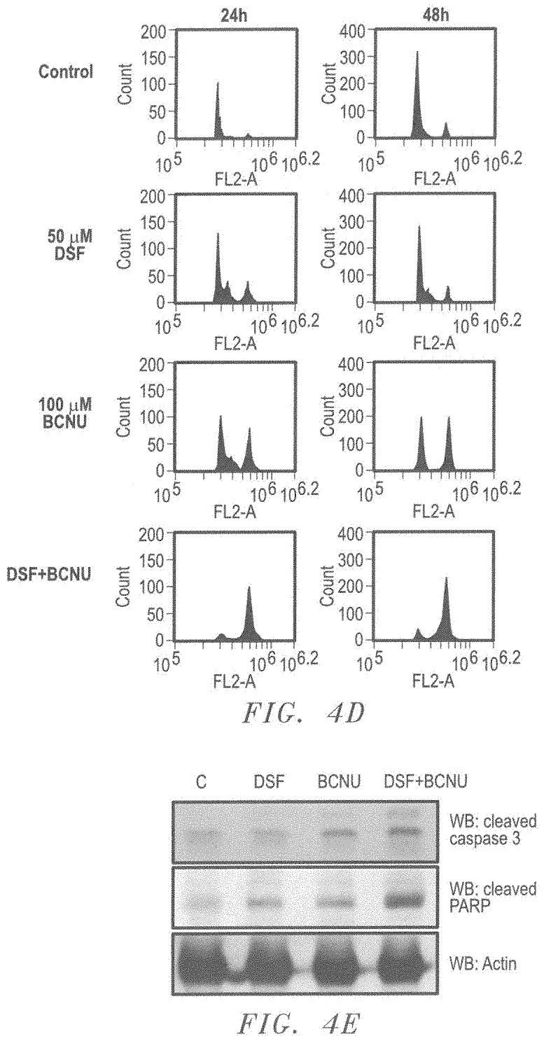

FIGS. 4A-4E show proteasomal degradation of DSF modified MGMT. FIG. 4A shows In vitro degradation of DSF treated rMGMT protein. 0.5 μg Unmodified rMGMT and DSF treated rMGMT (after dialysis) proteins were subjected to ATP and Mg2+-dependent degradation in rabbit reticulocyte lysates for 0-40 min as described in Methods. The samples were immunoblotted and probed with MGMT antibodies (right panel). The densitometric quantitation of protein bands on the western blots is shown as a line graph on the left. FIG. 4B shows the proteasome inhibitor PS-341 curtails the degradation of DSF-modified MGMT protein in brain tumor cells. UW228 cells were treated with 10 μM PS341 alone for 6 hours (lane 2), with 50 μM DSF alone for 12 hours (lane 3), pretreated 6 hours with 10 μM PS341 followed by 50 μM DSF for 12 hours (lane 4) and co-incubated with PS341 and DSF for 6 hours (lane 5). Cell lysates were western blotted for the MGMT protein. FIG. 4C shows DSF increases the DNA damage induced by MGMT-targeted alkylating agents. Kinetics of DNA interstrand crosslinks formed in UW228 cells after treatment with 100NM BCNU or 100NM melphalan is shown. Melphalan does not produce 06-alkylguanines, and served as a control. Cells were treated or untreated with 50 μM DSF for 12 hours to deplete the MGMT protein. They were then exposed to BCNU or melphalan. At times specified, the cells were harvested, DNA isolated and the extent of interstrand crosslinking of DNA was determined using the ethidium bromide fluorescence assay. Values are mean+S.D. The results were significant at P<0.05. FIG. 4D shows the effect G2/M blockade induced by BCNU is enhanced and extended in the presence of DSF. Histograms showing results of FACS-based cell cycle analysis of UW228 cells treated with BCNU or DSF+BCNU. Unsynchronous cultures were treated or untreated with DSF (50 μM for 12 hours) and then incubated with 100 μM BCNU. The cells were trypsinized at times indicated and analyzed by flow cytometry. FIG. 4E shows Enhanced levels of apoptosis markers—cleaved caspase 3 and cleaved PARP after DSF+BCNU treatments compared with BCNU alone.

Since the MGMT inactivated by BG is degraded through ub-proteolysis, it was of interest to investigate the mode of elimination of DSF-modified MGMT protein. Therefore, first, we compared the in vitro degradation of DSF-treated and untreated rMGMT protein in rabbit reticulocyte lysates (RL), which are known to promote the proteasomal degradation. The control and DSF-modified MGMT proteins were incubated with RL in the presence of Mg++, ATP and ubiquitin at 37° C. The reaction mixtures were blotted and probed with MGMT antibodies. The resulting western blot and the densitometric quantitation of protein bands are shown in FIG. 4A. The data shows that the untreated rMGMT remained largely undegraded during the 40 minutes incubation, whereas the DSF-modified protein disappeared gradually, 30% at 10 minutes, 45% at 25 minutes and 70% at 40 minutes of incubation (FIG. 4A). To show the involvement of the ub-proteolysis in cells a clinically used proteasome inhibitor. UW228 cells were treated with PS-341 alone for 6 hours, DSF alone, exposed to DSF after PS341, or co-incubated with PS341 and DSF, and the lysates were processed for western blotting of MGMT. PS341 co-incubation with DSF decreased the MGMT degradation by just 20%, whereas the cells pre-exposed to PS341 and then to DSF, showed a >50% reduction in the loss of MGMT protein (FIG. 4B, lanes 4-5). Taken together, the results presented in FIGS. 4A-4B confirm that DSF-modified MGMT is perceived as a structurally altered/damaged protein in cells and eliminated through the ub-proteasomal route.

As discussed in the Introduction, the inhibition of MGMT-mediated DNA repair is expected to augment the levels of alkylation DNA damage and the interstrand crosslinking of DNA induced by bifunctional alkylating agents. This hypothesis was tested by treating the UW228 cells with DSF for 12 hours followed by BCNU or melphalan. An ethidium bromide fluorescence assay was used to determine the levels of cellular DNA crosslinking. Cells treated with DSF and BCNU showed an approximately 2-fold increase in interstrand crosslinks as compared to BCNU alone (FIG. 4C). However, DSF was unable to increase the crosslinks generated by melphalan. While BCNU introduces the chloethyl groups to the O⁶-position of guanine, which are substrates for MGMT, melphalan is not an O⁶-guanine alkylator, but predominantly generates N7-guanine alkylations; thus, MGMT does not interfere with the damage induced by melphalan, and this explains the inability of DSF to enhance the melphalan-induced DNA crosslinking.

To determine the consequences of MGMT inactivation by DSF on cell cycle changes induced by BCNU, we performed flow cytometry (FIG. 4D). UW228 cells were treated with DSF alone, BCNU alone and a combination of DSF and BCNU, and histograms were generated from propidium iodide-stained cells. The results indicate that 50 μM DSF alone did not induce any cell cycle changes after 48 hours. Treatment with BCNU, as expected, produced a significant G2/M blockade (33% of cells) at 24 hours. Further, the cells pretreated with DSF and then exposed to BCNU showed a very strong accumulation of cells (˜80%) in G2/M phase at 24 hours, and this blockade was maintained at the same level at 48 hours (FIG. 4D). In DSF-treated UW228 cells, the greater level of DNA damage induced by BCNU (FIG. 4C) and the resulting lengthy cell cycle arrest (FIG. 4D) were associated with the activation of apoptotic machinery as reflected by an increased expression of cleaved caspase 3 and cleaved PARP proteins (FIG. 4E). Collectively, the evidence provided so far, distinctly indicates that the nontoxic drug DSF, acting through the inhibition of MGMT, is highly capable of potentiating the cytotoxic effects of alkylating agents.

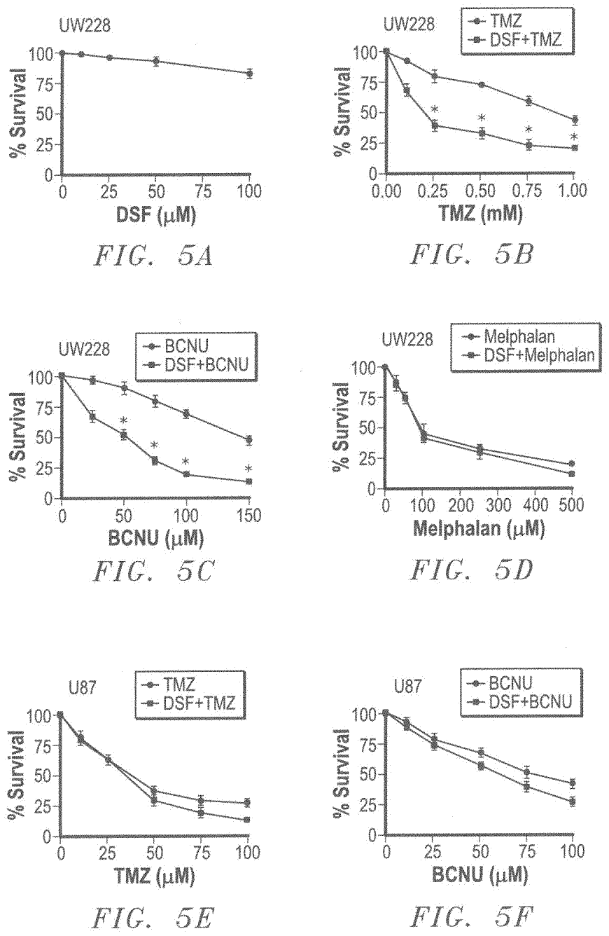

FIGS. 5A-5G show DSF preexposure sensitizes the brain tumor cells to the clinically used alkylating agents. FIG. 5A shows cell survival after DSF treatment alone. UW228 cells were treated with increasing concentrations of DSF (1-100 μM) for 24 hours. Cells were then cultured for 48 hours before performing the MTT assays. Since a 50 μM concentration for DSF generated a less than 5% cell killing, this concentration was chosen to potentiate the alkylating drugs. FIG. 5B shows TMZ mediated cell killing with and without DSF preexposure in UW228 cells. FIG. 5C shows BCNU mediated cell killing with and without DSF preexposure in UW228 cells. FIG. 5D shows DSF did not increase the cytotoxicity of melphalan, an N7, but not an O⁶-alkylator of guanine in UW228 cells. FIG. 5E shows DSF did not potentiate the TMZ induced cytotoxicity in the MGMT-deficient U87 malignant glioma cells. FIG. 5F shows DSF did not potentiate BCNU-induced cell killing in the MGMT-deficient U87 cells. In all these cases, the tumor cells were treated or untreated with 50 μM DSF for 12 hours followed by the alkylating drugs for 4 days before the MTT assays. FIG. 5G shows soft agar colony formation assay for cell survival analysis. UW228 cells were treated or untreated with 50 μM DSF prior to BCNU or TMZ exposures as described in Methods. The effect of drugs on cell survival was computed at each concentration. The data represents the results of three independent experiments performed in triplicate. Values are mean+S.D. The results presented in panels B, C and G (at 25-75 μM BCNU and 250-750 temozolomide) were significant at P<0.05. *, indicates statistically significant difference as compared to controls.

DSF can increase the efficacy of many anticancer drugs. In the context of this study, we performed cell survival assays using the MTT to determine the impact of DSF on alkylator-mediated tumor cell killing. DSF by itself, over a concentration range of 10-100 μM for 24 hours showed no toxic effects on UW228 (FIG. 5A) and other tumor cells (not shown). Based on this, we choose 50 μM DSF preincubation for 12 hours to test the potentiation of alkylating drugs. In this setting, TMZ (0-750+DSF combination showed a 4 to 5-fold increased cytotoxicity compared to TMZ alone (FIG. 5B). For BCNU, the potentiation was 3-fold (FIG. 5C). To confirm the MGMT-specific effects of DSF in cell survival assays, we used two types of controls. First, the drug melphalan, which does not generate O⁶-alkylguanines was used and DSF did not increase the melphalan-induced cell killing (FIG. 5D). Second, the effects of TMZ and BCNU were tested in DSF-pretreated U87 malignant glioma cells, which lack MGMT expression due to promoter methylation. DSF did not increase the extent of cell killing by TMZ or BCNU in U87 cells (FIGS. 5E-F).

Colony formation assays on soft agar were also carried out to confirm the DSF-induced increases in tumor cell killing. DSF treatment of UW228 cells followed by BCNU or TMZ exposures resulted in a 3-fold enhanced cytotoxicity for both drugs (FIG. 5G). The cell survival assays, again, demonstrate that MGMT inhibition by DSF is a significant factor in amplifying the cytotoxic effects of the clinically used alkylating agents.

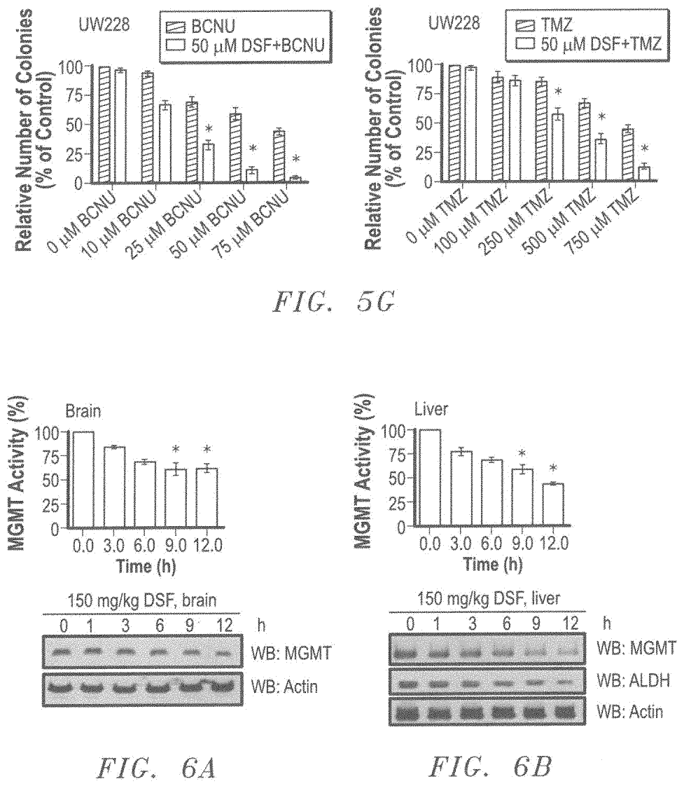

FIGS. 6A-6C show significant downregulation of MGMT activity and protein in mouse brain and liver tissues after DSF administration. Animals in groups of six were given i.p. injections of a single DSF dose (150 mg/kg). The mice were sacrificed at 3, 6, 9 and 12 hours post-DSF. Liver and brain tissue lysates were prepared as described in Methods and used for MGMT activity assays and western blotting. FIG. 6A shows DSF treatment resulted in a sustained 40% inhibition of brain MGMT activity starting at 9 hours (upper panel). Western blot analysis (lower panel) shows a gradual decrease of MGMT protein in the same time course. FIG. 6B shows decreased hepatic MGMT activity (60% inhibition, upper panel) and protein levels (lower panel) after DSF administration. The decrease of MGMT activity in DSF-treated animals in both liver and brain were significant as compared to the values in mice treated with the vehicle alone. *, P<0.05 compared with controls. Error bars indicate SD. Western blot analysis was also performed for ALDH (a major target of DSF) in liver lysates, and decreased protein levels are evident. FIG. 6C is a scheme showing the consequences of MGMT inhibition by DSF and increased in tumor sensitivity to alkylating agents. The overall findings of the present study are summarized. The active site cysteine (Cys145) of MGMT which accepts the alkyl groups in a self-inactivating reaction is highly reactive. The electrophilic DSF and its metabolites can form adducts with this cysteine and inactivate the MGMT-catalyzed removal of alkylation DNA damage. Subsequent administrations of alkylating agents induce higher levels of the cytotoxic 06-alkyguanine lesions.

To further illustrate MGMT inhibition by DSF in a preclinical setting, a single dose of DSF (150 mg/kg, i.p.) was administered and the liver and brain tissues isolated 1, 3, 6, 9 and 12 hours post-injection, and determined MGMT activity in clarified tissue lysates. MGMT activity in 6 animals at each time point was averaged and used to compute the changes in DNA repair activity. There was a gradual reduction of MGMT in brain with a 40% decrease of the repair activity occurring at 9-12 hours (FIG. 6A, upper panel). The MGMT protein levels correlated well with the observed changes in the activity (FIG. 6A, lower panel). In liver tissues, MGMT inhibition was much stronger with a 60% decrease observed at 12 hours relative to the controls (FIG. 6B, upper panel). Hepatic MGMT protein appeared to undergo degradation in DSF treated animals, in a manner similar to that observed in cancer cell lines (FIG. 6B, lower panel). Significantly, the hepatic levels of ALDH protein, the primary target of DSF mediating the alcohol antagonism, also showed a progressive decline in DSF-treated animals, much similar to that observed for the MGMT protein. To our knowledge, this is the first report showing a decrease of ALDH protein in DSF-treated animals. Overall, the data obtained in animal studies establish that DSF can curtail the MGMT activity in the brain and strengthens the drug's utility in brain tumor therapy.

Recently, as a part of an international effort, we proposed that a cocktail of 9 pharmacologically well-characterized nontoxic drugs, collectively called CUSP9 be added to a continuous low dose temozolomide for improving survival and quality of life for relapsed glioblastoma patients, who have one of the worst 5-year survival rates among all human cancers. The nine adjuvant drug regimen consists of the aprepitant, artesunate, auranofin, captopril, copper gluconate, disulfiram, ketoconazole, nelfinavir, and sertraline. Of these drugs, disulfiram has the strongest evidence of potential benefit in CNS cancers. The copper gluconate in the regimen is meant to enhance the efficacy of DSF, because the copper bound drug has been shown to be more potent and exert higher levels of cytotoxicity. In fact, the ongoing clinical trials of DSF in metastatic cancers of the liver, prostate, and melanoma include the copper gluconate or zinc gluconate as a component. Consistent with this premise, we found that copper-chelated DSF was about 5-fold more potent than DSF in inhibiting the MGMT activity in cultured brain tumor cells (FIGS. 1B, 1C).

One embodiment of the present invention is summarized in FIG. 6C. Chemically, DSF has a symmetrical structure and its first metabolic step is the reduction of the disulfide group at the center of the molecule to yield two diethyldithiocarbamate (DDC) moieties. DDC is further converted to its methyl ester, and other metabolites. DDC is a potent inhibitor of ALDH, forming mixed disulfide bridges with a critical cysteine near the active site. Our data from the cell culture and animal studies indicate that DSF interacts with Cys145 of MGMT the same way to inactivate the DNA repair protein (FIG. 6C). The covalent adducts induced by DSF alter the secondary and higher order structure of the MGMT protein, allowing the inactivated protein to be recognized by the ubiquitin conjugating enzymes. Evidence clearly pointing to the involvement of the ubiquitination-dependent proteolysis in the processing of DSF-conjugated MGMT was obtained (FIG. 4A, B). Our recent findings that DSF mediates the degradation of several redox-sensitive proteins (p53, NF-κB, ub-activating enzyme E1) in tumor cells clearly agree with the observations made here. The covalent adduct introduced by DSF is non-reducible at physiological concentrations of GSH, and actually, the endogenous GSH has been reported to increase the inactivation of ALDH by DSF metabolites. Similarly, it was found that MGMT inactivation by DSF was not reversible by thiols (DTT or GSH).

One surprising and unexpected result was that DSF has MGMT-dependent biochemical effects. Also clear from the literature is that DSF has multiple cellular targets and promotes the efficacy of anticancer agents of different classes in a pleiotropic “MGMT independent” manner as well. We showed potent inhibition of MGMT activity and the resulting protein degradation in tumor cell lines and animal tissues. BG, the MGMT inhibitor in clinical trials, has also been shown to inhibit MGMT in normal tissues in animals and humans. DSF, in contrast to BG, appeared to be a weaker inhibitor of MGMT causing a short-term inactivation of MGMT>30 hours, which is still sufficient for enhancing the efficacy of alkylating agents, because, the alkylation of DNA is a rapid process. The promotion of BCNU-induced DNA crosslinking in DSF treated cells (FIG. 4C) again supports this postulate. Moreover, the faster regeneration of MGMT following DSF treatment is likely to decrease the toxicity to the normal tissues such as the marrow.

From the discussion above, it is clear that DSF has a huge potential for therapy of CNS tumors. It is a drug already approved by regulatory agencies for human use, its pharmacokinetics and safety issues are well known; this should enable a faster clinical application. DSF has a favorable lipophilic profile (Molecular weight 296.5 Daltons, partition coefficient Log P of 2.8) to cross the blood-brain barrier, and many studies have established its entry in to the brain. Further, disulfiram administered in the absence of alcohol is largely non-toxic and tolerated very well. Although no precise data is available in humans, DSF can be given up to 1 g/day in adjuvant settings to non-alcoholic patients. In rats, DSF at high doses of 600 mg/kg daily for 3 days or smaller doses (up to 100 mg/kg) at twice a week dosing has been shown not to exert any liver toxicity. Also noteworthy is that DSF appears to be selectively toxic to human cancer cells compared to the normal cell counterparts; thus, in pairs of the chronic lymphocytic leukemia and normal lymphocytes, invasive cancer and normal endothelial cells, the glioblastoma and normal astrocytes, there was a preferential killing of cancer cells by DSF. The redox-sensitive nature of the human MGMT protein was exploited and since DSF is (i) nontoxic, (ii) is hydrophobic enough to cross the blood-brain barrier, (iii) unlikely to induce tumor resistance, (iv) has MGMT-independent signaling effects that may actually promote the chemotherapy, the ability of DSF to create an MGMT-deficient state provides for improving the brain tumor therapy.

In addition, the present disclosure shows that chronic alcoholics undergoing disulfiram therapy for a long time are likely to have an increased risk for developing cancer. This is because, alcohol, by itself is known to downregulate the MGMT activity. DSF, through a direct effect reported here, can further exacerbate the MGMT inactivation, and the ability of different organs to defend themselves against the endogenous and environmentally derived alkylating agents may be compromised. Such an unrepaired DNA damage, particularly in the regulatory oncogenes and tumor suppressor genes, may manifest in harmful mutations and promote the genomic instability. The reported occurrence of frequent mutations in K-ras, p53 and β-catenin genes in cells with reduced MGMT activity or silencing of the MGMT gene through promoter methylation in human cancers is consistent with this premise.

NCX-4016 (NO-Aspirin), 2-(acetyloxy) benzoic acid 3-[(nitrooxy) methyl]phenyl ester, is an acetylsalicylic acid molecule linked at the meta position to a chemical spacer (hydroxybenzylalcohol) bearing the NO-donating moiety (benzenemethanol-3-hydroxy-d-nitrate or NCX 4015). It is metabolized in the liver and plasma by esterases to yield the parent drug and a (nitromethoxy) phenol which is the spacer attached to the NO releasing moeity. Further biotransformation of the (nitromethoxy) phenol yields NO-3 at a much slower rate. Reactive (ionized) cysteines present on protein surfaces perform essential functions and are preferentially attacked by ROS and RNS. The consequences of cysteine oxidation and protection against these oxidations by reversible and dynamic glutathionylation and nitrosylation, and their interrelationships are shown. MGMT is overexpressed in human cancers and thereby decreases the formation of mutagenic 06-alkylguanine adducts and cytotoxic DNA interstrand crosslinks. Currently, 06-benzylguanine (BG), a potent inhibitor of MGMT is in clinical trials for increasing the efficacy of alkylating agents. However, this strategy is beset with the development of BG resistance, and cumulative myelosuppression. The compounds were tested which posttranslationally modify the active site Cys145 (a reactive cysteine) as a new strategy for powerful inhibition of MGMT.

Nitrosylation or covalent modification of reactive cysteines present in many cancer chemotherapy target proteins was designed as a new anticancer strategy. Two non-toxic drugs, namely, disulfiram (a thiolating agent) and nitroaspirin (nitrosylating agent) were used in our approach. Most significantly, nitroaspirin caused a rapid inhibition and degradation of the DNA repair protein MGMT at physiologically achievable concentrations. The extent of inhibition and elimination of MGMT protein by nitroaspirin was well-comparable with those achieved by 06-benzylguanine (BG), an MGMT inhibitor currently undergoing clinical trials. Therefore, the modulation of the active-site Cysteine 145 (pKa=6.4) of MGMT by redox-modifying compounds will provide new and superior therapeutic avenues for enhancing the antitumor efficacy of clinically-used alkylating agents, without the harmful bone-marrow suppressive effects seen with the BG+alkylator strategy. A dramatic and dose-dependent degradation of many redox-regulated proteins (p53, GST-π, NFκB, MGMT, and p21cip1) by disulfiram was also observed.

Synthesis, characterization, cellular and preclinical studies of KSS-72, a non-diuretic analog of ethacrynic acid (EA) for pediatric brain cancers: Ethacrynic acid [Edecrin, (2,3-Dichloro-4-[2methylenebutyryl] phenoxy)acetic acid] is a potassium-sparing loop diuretic used in the clinic for hypertension and edema treatment. The carboxylic acid side chain confers the diuresis property. The inventors removed this group to prepare a hydrophobic non-diuretic analog called KSS-72. Compared to EA, KSS-72 was 15 times more potent in inhibiting the GST-pi and conjugating with glutathione to induce ROS, consequently inhibiting the MGMT, altering the mitochondrial membrane permeability, and triggering apoptosis. The astrocytes and normal cells were not affected. Pharmacokinetics of KSS-72 after either IV injection or oral gavage showed that it enters the brain easily, accumulates effectively in the CNS at 20-22% of plasma concentrations, equivalent to that of temozolomide, (TMZ), a standard anti-glioma drug. In nude mice, the intracranially implanted DAOY medulloblastoma, SF-188, and T98G GBMs were eliminated after 25 days of IP or oral gavage treatments at 25 mg/kg. There were no signs of bone marrow suppression and organ toxicities. To make KSS-72 pediatric compliant, the inventors have prepared oral suspensions in grape and cherry flavors (5 mg/ml), where the drug remained stable and showed the same levels of antitumor efficacy and excellent synergy with TMZ in orthotropic GBM xenografts.

Synthesis of 2,4-Dimethylene glutaric acid (DMG), an α-ketoglutaric acid (α-KG) mimic for induction of epigenetic modifications: D-2 hydroxyglutarate (D-2HG) effectively replaces the normal metabolite, α-KG in 70% of low-grade pediatric gliomas with IDH mutations. α-KG is a substrate for TET1, TET2 DNA demethylases, and histone demethylases that maintain normal epigenetic homeostasis. D2-HG directly contributes to the CpG-island methylator phenotype (G-CIMP) and MGMT promoter methylation by competing with α-KG and inhibiting the demethylases. As an innovative strategy of understanding the glioma pathology and turning the tide against it, the inventors prepared DMG, a powerful α-KG derivative to replace the natural metabolite in epigenetic demethylase reactions; induction of such therapeutic methylation in non-IDH mutant GBMs was postulated to regress the tumors. DMG indeed curtailed TET1, MGMT activities, induced de novo histone methylations, and potent oxidative stress in GBM cell lines. A 6-8 fold potentiation of TMZ was seen in DMG-treated cells. DMG efficacy in xenografts and MGMT methylation therein are being tested.

DSF nanoparticles (NPs), uptake, and efficacy: the inventors showed disulfiram is a direct inhibitor of MGMT modifying the active site Cys145, DSF nanoparticles made in PEG/PLGA matrices were found to be taken up by the brain and internalized to trigger apoptosis. The NPs had a long half-life, caused no toxicity, and showed great efficacy in intracranial GBM.

Human MGMT is an S-phase checkpoint, and its inhibition sensitizes gliomas to antimetabolites:

MGMT performs stoichiometric catalysis to remove mutagenic alkyl groups and dies in the process. The inventors showed that MGMT has other functions in the cell cycle by interacting with PCNA, p21cip1, and replication licensing components (CDT1 and geminin) in restricting the genome replication to occur only once in the cell cycle (Neoplasia 20, 305, 2018). Inhibition of MGMT by siRNA or 06-benzylguanine led to an S-phase block, perturbed DNA synthesis, and re-replication of the genome. In support, the inventors showed antimetabolite drugs like 5FU and Ara-C were >10-15 fold more cytotoxic in MGMT inactivated cells and xenografts due to mitotic catastrophe.

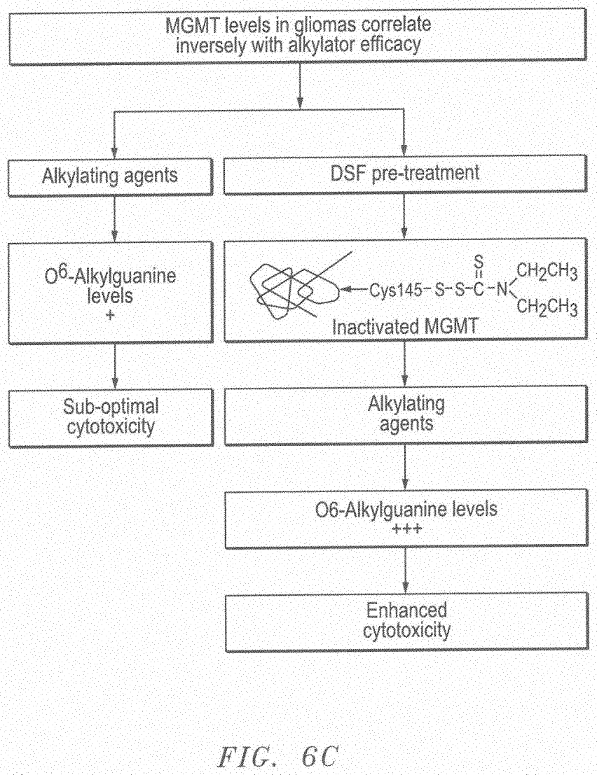

DSF is unstable and is cleaved into two diethyldithiocarbamate (DDC) moieties, which if they encounter Cu(II) ions, form a stable coordinate complex called Cu-DDC (FIG. 7). Cu-DDC, a lipophilic water-insoluble black compound is the ultimate cytotoxic activity of DSF.

FIG. 7 shows disulfiram, decomposition, and Cu-DDC formation. DSF is a symmetrical compound, is reduced and split in the middle to yield two dithiocarbamate (DDC) groups in the GI tract or blood. If copper ions are available, a stable coordinate complex Cu-DDC is formed. The physical appearance of Cu-DDC as fine black particles is shown on the left. If Cu is not around, part of the DDC undergoes methylation (active component) forming adducts with active site cysteines of enzymes. NPL4 is a protein important in the processing of ubiquitinated proteins was shown to be a direct target of Cu-DDC recently (Nature, 2017 Dec. 14; 552(7684):194-199, 552, pages 194-199 (2017)), the resulting proteostatic effects are listed.

DSF as a successful anticancer drug is hindered by the super instability in both the acidic gastric environment and the bloodstream, compromising the in vivo antitumor effects through degradation into metabolites without cell killing activity. Further, the chances of a toxic metabolite being formed and reaching the tumors with the current approach of separate DSF and Cu-gluconate administrations is extremely small. This is the major reason why limited success was reported in a recent clinical trial of DSF and Cu-gluconate in newly diagnosed glioblastoma. In contrast to DSF, Cu-DDC is a stable chemical with a longer half-life, and we will develop this compound as an anticancer agent for the first time. To overcome (i) any toxicity Cu-DDC may have and (ii) to facilitate a preferential accumulation of this insoluble compound in malignant tissues, the inventors can engineer serum albumin nanoparticles laden with the drug and test the efficacy in orthotopic tumor models. This delivery system exploits the Enhanced Permeability Retention (EPR) properties of tumors and the overactive albumin receptor (gp60)-mediated trans-endothelial transport and SPARC proteins highly expressed in the tumor microenvironment.

NQO1 and NQO1-targeted therapeutics: Human NAD(P)H quinone oxidoreductase-1 (hNQO1), formerly known as DT diaphorase, is a multifunctional cytosolic flavoenzyme that protects cells against endogenous quinones in a two-electron transfer reaction.

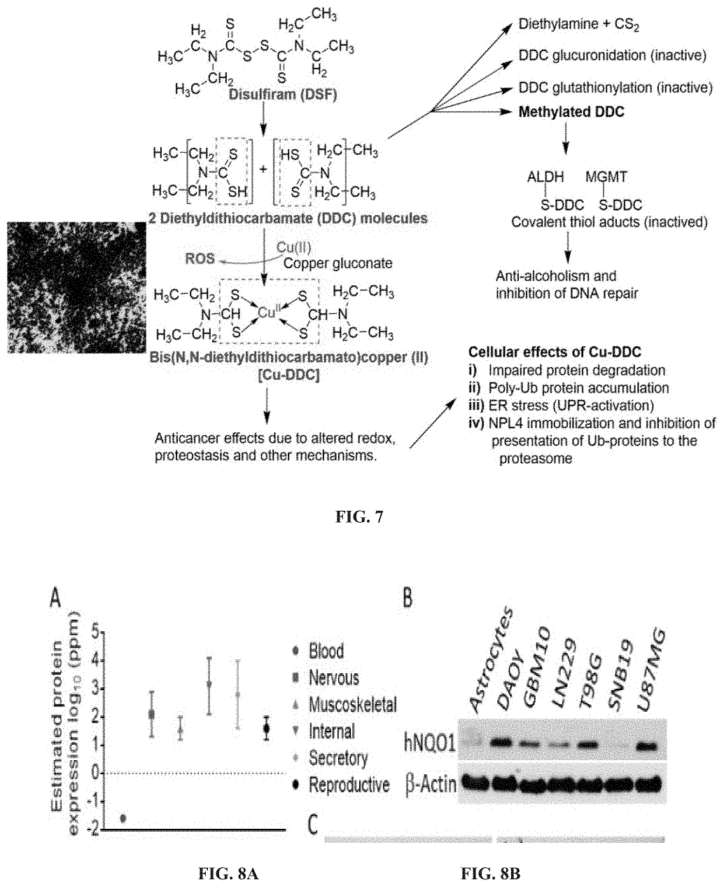

FIGS. 8A to 8C show, elevated expression of NQO1 in human cancers (FIG. 8A); absence of NQO1 in human astrocytes and its abundance in brain cancer cell lines (FIG. 8B); IHC staining of GBM-12 tumor- and β-lapachone) to cytotoxic agents either through DNA alkylation or production of ROS through futile redox cycling (FIG. 8C). NQO1 is constitutively expressed at low levels in various normal tissues but is frequently expressed at very high levels (10-200-fold) in most solid tumors including brain tumors. Additionally, the glioma tumor suppressor PTEN has been shown to regulate the expression of the NQO1 gene. NQO1mRNA and protein levels up to 50-fold higher have been quantitated in tumor tissues relative to the normal cell counterparts.

Furthermore, human tumors are deficient in antioxidant enzymes such as catalase and superoxide dismutases relative to normal tissues, which serves to exacerbate the levels of redox stress in tumors. The increased expression of NQO1 represents an adaptation response to the increased oxidative stress inherent in malignant tissues and is mediated by the NRF2 transcription factor. NRF2 dissociates from the KEAP inhibitor in redox-perturbed malignant cells and upregulates various cytoprotective enzymes. NQO1 is a prototype target gene for NRF1 and NRF2 because these transcription factors bind to multiple copies of antioxidant response elements (ARE) present in the 5′ regulatory region of the gene and activate NQO1 transcription. By way of explanation, but in no way a limitation of the present invention, augmented NQO1 expression provides a means to generate more ROS and damage using bioactivatable quinone substrates selectively in tumors for chemotherapy. The higher NQO1 content is also closely linked with tumor aggressiveness, chemotherapy resistance, and poor prognosis. As such, NQO1 has emerged as a cancer biomarker; the specific and non-invasive detection of NQO1 activity as shown here is highly desirable to improve the diagnostic efficacy and predict positive responses to NQO1-bioactivated drugs.

Therefore, the NQO1 enzyme, which is inducible by redox stress and present abundantly in brain cancers, but not in normal brain, was targeted. NQO1 can bind certain quinone substrates and reduce them to unstable hydroquinones, which in turn react with molecular oxygen to produce superoxide and regenerate the quinone. Because this catalytic redox cycling generates large amounts of ROS and H2O2 only in NQO1-expressing cells, the bioactivatable compounds bear a high potential for tumor-selective therapy.

Hitherto, only the 3-lapachone and deoxynyboquinone have been characterized as prototype NQO1 bioactivators. Both of them have solubility and structural problems (cannot be derivatized) and cause hemolytic anemia.

The present inventors developed synthetic Cu-DDC that is directly, potently, and preferentially cytotoxic to glioma cells in culture at nM concentrations and eliminates the intracranial implants of medulloblastoma and GBM cells. Cu-DDC generated only peroxides but not superoxides. p53 gene status was irrelevant in this cell killing as both the wild-type and gain-of-function (GOF) mutant cells (R273H, R248W) underwent massive apoptosis in the absence of discernible adverse effects. Unexpectedly, the Cu-DDC induced a rapid degradation of the oncogenic p53 mutant proteins in cells, thus, providing an added advantage to the therapeutic strategy. Cu-DDC encapsulated in serum albumin nanoparticles and DDC prodrugs that are designed to selectively accumulate in cancers exerted potent cytotoxicity in our in vitro and in-vivo models. In search of new generation NQO1 bioactivators with greater specificity, the inventors have designed and synthesized a BBB-permeable NQO1-directed spiroisoindolinone called GNQ and NQ-IND (a derivative of ES936, a proven inhibitor of NQO1) and established the role of NQO1 in their bioactivation. GNQ showed a striking ability to eliminate the intracranial GBM and medulloblastoma in nude mice. Compounds that became highly fluorescent in the infrared absorption range specifically after NQO1 catalysis have been developed and their application to image the NQO1-positive but not the NQO1-negative tumors located in live nude mice within minutes after IP injections has been demonstrated. Potent inhibition of the DNA repair protein MGMT and the mediation of tumor cell death by ROS, ER stress, and release of DAMP (damage-associated molecular pattern) molecules indicative of immunogenic cell death by GNQ.

The inventors found that (i) Cu-DDC triggers cell death through unique redox-alterations, injectable Cu-DDC trapped in human serum albumin (HSA) nanoparticles (NPs) that preferentially accumulate in childhood brain cancers through a leaky tumor vasculature, and overactive albumin receptor-mediated influx, and that prodrugs of diethyldithiocarbamate (DDC) in which the DDC groups remain stable, masked and unreactive until cleavage by tumor enriched enzymes/pro-oxidative setting will selectively accumulate in CNS cancers; oral administration of Cu-gluconate, in this case, leads to tumor-selective Cu-DDC production; and (ii) that NQO1 bioactivatable drugs uniquely exert tumor-selective cytotoxicity against children's CNS cancers with minimal toxicity to normal tissues, the consequent DNA repair defects (MGMT, PARP activation) can be targeted for combination chemotherapies and that NQO1 imaging makes them useful for fluorescence-guided surgery.

Intracranial glioblastoma xenograft development in nude mice: GBM cells stably expressing luciferase and GFP are suspended at 2×10⁵ cells/5 μl sterile PBS injected into mouse brains. For developing athymic mice, mice are anesthetized and placed on a stereotactic apparatus. For developing breast cancer brain metastases xenografts, brain-seeking breast cancer cells are injected into the left ventricle of the heart of each mouse under anesthesia. Three weeks later the mice are imaged for intracranial Firefly luciferase activity. Mice with intracranial bioluminescence are randomly divided into different groups for drug treatments. Imaging is performed 10 minutes after i.p. injection of D-luciferin (2 mg per animal) with mice lying in the prone position. Besides the bioluminescence studies, the survival of the human glioblastoma cell grafted mice after drug administrations are closely followed to generate the Kaplan-Meir curves.

Glioblastoma organoids (GBO) for drug sensitivity analysis: GBOs are established from glioblastoma specimens from pediatric patients and used for testing the efficacy of Cu-DDC nanoparticles, DDC prodrugs, and NQO1-targeted drugs. Primary cultures of GBM organoids of 300-1000 μm are set up by mechanical dissociation of tissue followed by self-aggregation in short-term cultures, up to 2 weeks.

These cultures represent self-organizing structures, which preserve a heterogeneous 3D tumor tissue organization, inter and intra-tumor heterogeneity, cell-cell interactions, non-neoplastic cells of the tumor microenvironment, extracellular matrix, and original hypoxic conditions. After exposure to different drug concentrations and continued culture, confocal microscopy is performed to assess changes in size. At the end of the experiments, the GBOs are stained for the expression of the Ki67 proliferation marker, and the proliferation index is computed.

Cu-DDC is used as a free compound showed potent cytotoxicity at sub-micromolar concentrations against human cancer cell lines including brain cancers (U87MG, SNB19), while the normal cell representatives were significantly resistant (30-50 fold) as shown in Table 1.

Table 1. DPSC—Dental pulp stem cells; HUVEC-human umbilical vein endothelial pediatric GBM cells.

Cytotoxicity of Cu-DDC against normal and cancer cells

| Cancer Cell line | P53 Status | IC₅₀ ± SD (μM) |
|---|---|---|
| MAI PaCa-2 | R248W | 0.5 ± 0.3 |
| HT29 | R273H | 0.66 ± 0.03 |
| SKBR3 | R173H | 0.32 ± 0.01 |
| SNB19 | R273H | 0.65 ± 0.01 |
| H1299 | Null | 0.31 ± 0.001 |
| HCT116 | Wild type | 0.25 ± 0.01 |
| U87MG | Wild type | 0.58 ± 0.01 |
| Representative normal cell type | | |
| IMR90 | Wild type | 17.2 ± 0.1 |
| DPSC | Wild type | 14.6 ± 0.7 |
| HUVEC | Wild type | 21.3 ± 0.7 |
| hPBL | Wild type | 15.1 ± 0.5 |

FIGS. 9A and 9B show the selective generation of peroxides but not superoxides by Cu-DDC in SF-188. Cells were treated with DCFDA (which detects ROS such as H₂O₂ and OH or DHE (dihydroethidium) which detects O2.— species followed by Cu-DDC (5 μM) for 1 h. Flow cytometry was performed to quantitate the fluorescence generated by the dyes. N-acetylcysteine and Tyron were used to quench the ROS.

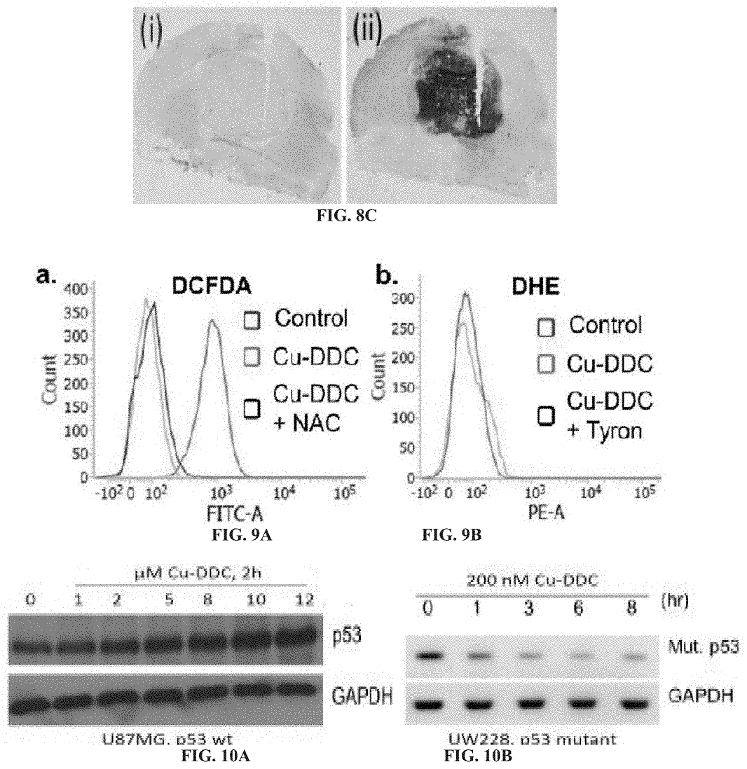

FIGS. 10A and 10B show that, while Cu-DDC upregulated the wt p53, it dramatically eliminated the mutant p53 found in UW228 medulloblastoma and other GBMs.

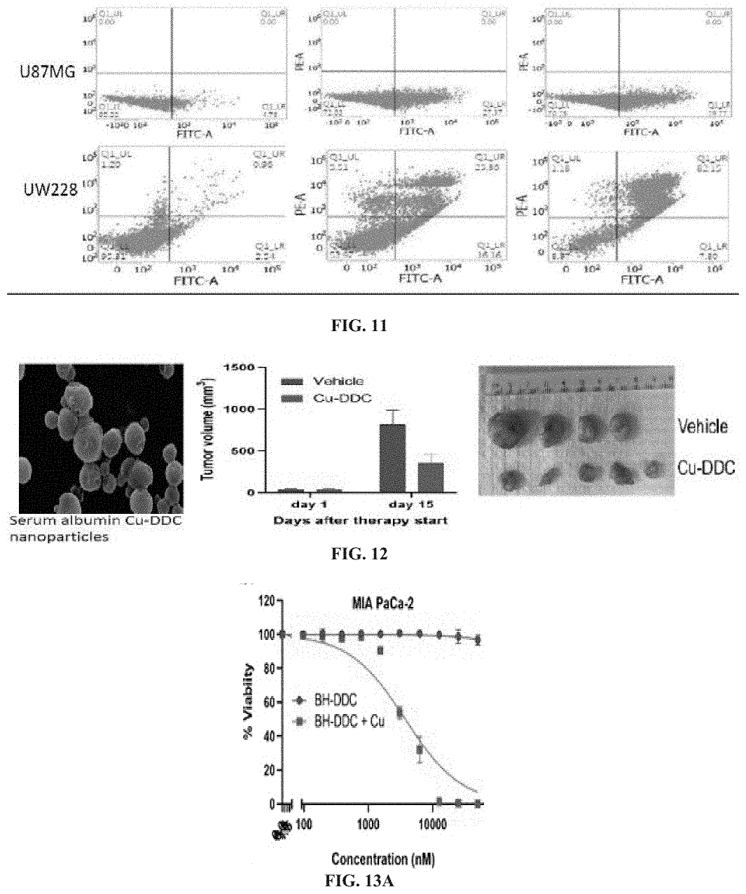

FIG. 11 shows that irrespective of the p53 status, Cu-DDC triggered dramatic cell death as detected by Annexin-V assay. The left most panel is control and the two right correspond to 10 and 20 h after Cu-DDC, 5 μM exposure.

FIG. 12 shows that BSA nanoparticles (NPs) loaded with Cu-DDC showed significant antitumor efficacy against the DAOY medulloblastoma orthotopic xenografts. Photograph of protein nanoparticles and significant tumor regression is evident. Nude mice bearing intracranial DAOY tumors were injected with NPs with Cu-DDC equivalent to 10 mg/kg twice a week for two weeks.

The inventors prepared medical-grade Cu-DDC protein nanoparticles of 130-150 nm. The rationale for designing protein nanoparticles for delivery is as follows. 1) Cu-DDC is a powdery highly hydrophobic and insoluble compound; it can only dissolve in organic solvents. 2) Although the free compound Cu-DDC is 15-30 times less toxic to normal cells (Table 1), it is possible that adverse effects can occur if direct drug suspensions are used. 3) There is a need for adequate delivery to tumors while sparing the normal tissues. 4) Serum albumin formulations, besides being physiological and biocompatible can act as decoy delivery vehicles exploiting the very same pathological properties tumors utilize for their survival.

The inventors prepared DSF-NPs before and characterized their physicochemical properties (18). Coacervation/desolvation is used for this process. A 20% BSA solution is mixed with the drug suspension followed by ethanol addition to achieve phase separation. These drug encapsulated formulations are crosslinked and stabilized with glutaraldehyde. for stabilization. An increase in the degree of crosslinking generally decreases the particle size due to the formation After freeze-drying, the aggregates are homogenized. The particles are analyzed for size distribution by dynamic light scattering (DLS), scanning electron microscopy, the zeta potential for determining the drug entrapment efficiency.

Cu-DDC-HSA nanoparticles can be used to deliver the insoluble drug to cancers. The antitumor efficacy obtained can be used in high-risk terminally ill patients.

Other biodegradable matrices such as the PEG: PLGA or tannic acid to engineer Cu-DDC nanoparticles can also be used. Further efficacy can be obtained to actively target nanoparticles includes decorating the nanoformulations with ligands or antibodies for specific tumor delivery.

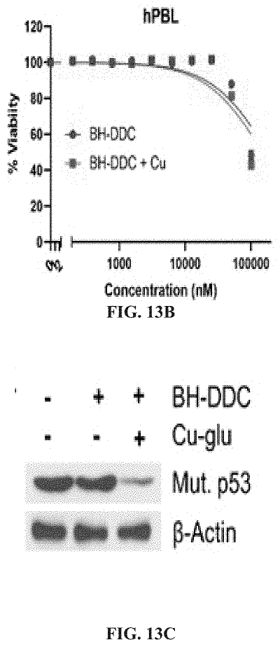

The inventors synthesized the boronic acid and lysine hybrids of the DDC. Boronic acid linkages are broken by H₂O₂ in a prooxidant tumor environment and lysine is detached by an aminopeptidase. Table 2 and FIGS. 13A to 13C show that cancer cells including the SNB GBM cells show a dramatic increase in cytotoxicity when the DDC drugs are combined with copper gluconate. For Mia PACA pancreatic cancer cells (FIG. 13A), the BH DDC+Cu achieved a 54-fold increased cell killing, whereas the cultured human peripheral blood lymphocytes (normal cell representative (FIG. 13B)) were not killed at all. Mia PACA pancreatic cancer proliferation is driven by mutant p53. The western blot (FIG. 13C) shows that Cu-DDC was able to degrade the gain-of-function p53 mutant protein (R248W) in Mia-PACA cells, halt the cell proliferation, and induce cell death, which attests to the huge potential of Cu-DDC in defeating the oncogenic pathways for therapeutic benefit, as proposed for pediatric brain neoplasms in this application.

Table 2 shows that cancer cells including the SNB GBM cells show a dramatic increase in cytotoxicity when the DDC drugs are combined with copper gluconate.

| | IC₅₀ (μM) ± SD | | | |
|---|---|---|---|---|
| Cancer | BH-DDC | BH-DDC + | Lys-DDC | Lys-DDC + |
| Cell line | alone | Cu | alone | Cu |
| MIA paCa-2 | >200 | 3.7 ± 0.3 | 47.0 ± 4.8 | 15.7 ± 1.6 |
| HT29 | 189 ± 8.2 | 2.9 ± 0.01 | 35.6 ± 4.6 | 10.5 ± 0.6 |
| SNB19 | 17.0 ± 0.2 | 1.1 ± 0.01 | 46.0 ± 3.2 | 9.6 ± 0.5 |
| HCT116 | 6.8 ± 0.5 | 0.3 ± 0.01 | 43.2 ± 3.8 | 2.3 ± 0.02 |

Representative normal cell type

| | | | | |
|---|---|---|---|---|
| HUVEC | 164 ± 6.3 | 92 ± 5.8 | 137 ± 12 | 85.9 ± 6.1 |
| hPBL | 182 ± 11 | 152.7 ± 5.8 | >200 | 153 ± 10 |

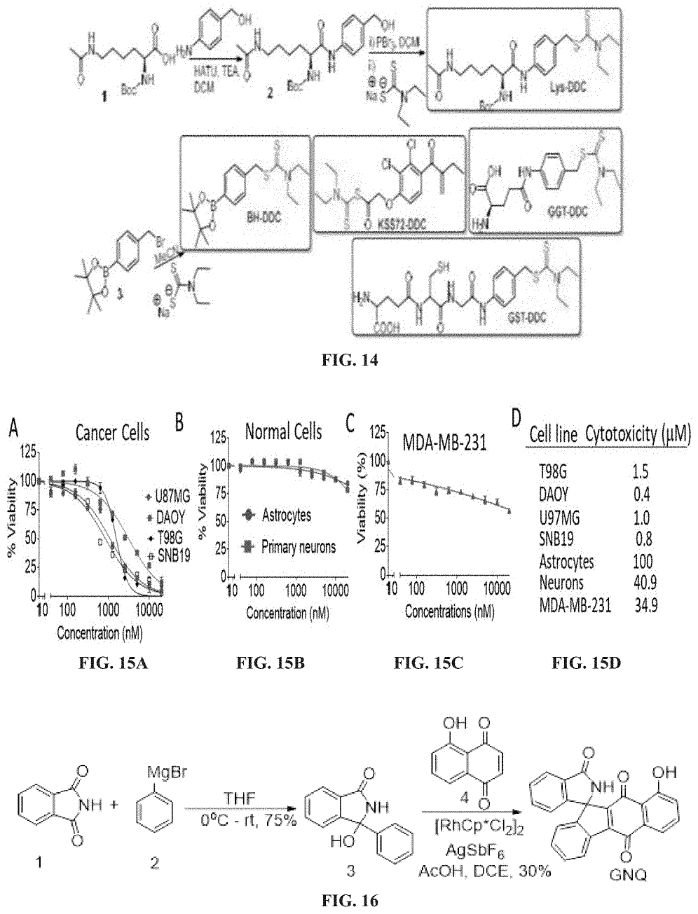

FIG. 14 shows the synthetic pathway for the various compounds of the present invention.

The inventors next looked at the pharmaco-biological, safety, and anti-cancer parameters of GNQ in pediatric brain cancer models and to develop more specific blood-brain-barrier (BBB)-penetrable NAD(P)H Quinone Dehydrogenase 1 (NQO1) bioactivatable substrates based on this drug.

The overexpression of NQO1 in malignancies and its almost absence in corresponding normal tissues and the ability of the enzyme to generate ROS from a unique class of rare quinones has been recognized as a pathway for tumor-selective cytotoxicity. However, this disclosure is the first one to propose NQO1 as a target for brain cancers and suggest the use of synthetic quinones.

Here, the substrate quinones are activated to generate ROS by the first reduction to their hydroquinones, which rapidly react with molecular oxygen in the cell to give two moles of superoxide and regenerate the original quinone.

The phytoquinones β-lapachone and deoxynyboquinone are known examples of the bioactivatable drugs which cause NQO1 dependent cytotoxicity. The ROS produced cause DNA single-strand breaks, hyperactivation of PARP-1, loss of NAD+ and ATP pools, inactivation of signal-transducing regulators, and programmed necrosis to initiate cell death.

In normal tissues, NQO1 is expressed constitutively and is never induced, thus largely restricting the oxidative damage to the malignant cells and sparing the normal counterparts. The plant quinones are highly insoluble and have to be formulated with hydroxypropyl-β-dextran, which itself was toxic. Their activity against brain cancers has not been investigated.

Therefore, the inventors developed a blood-brain barrier (BBB) permeable novel class of quinone, spiroisoindolinone (GNQ) as an NQO1 substrate for glioma therapy. It was found that GNQ is a superior substrate for NQO1 and demonstrated a strong correlation between NQO1 bio-reduction and anticancer activity.

FIGS. 15A to 15D show that: (FIG. 15A) Cytotoxicity of GNQ against different brain tumor cells. (FIG. 15B) Cytotoxicity of GNQ against normal cells astrocytes and primary neurons. (FIG. 15C) Cytotoxicity against NQO1 null cell line MDA-MB-231. (FIG. 15D) Cytotoxicity values against normal and cancer cells in micromoles. The cytotoxicity of GNQ was dependent on the presence of NQO1 in cells and the potent cytotoxicity of GNQ was reversed by coincubation with NQO1 inhibitors ES936 or dicumarol. Normal astrocytes and NQO1-null cells were not sensitive to GNQ.

FIG. 16 shows the synthetic pathway for GNQ.

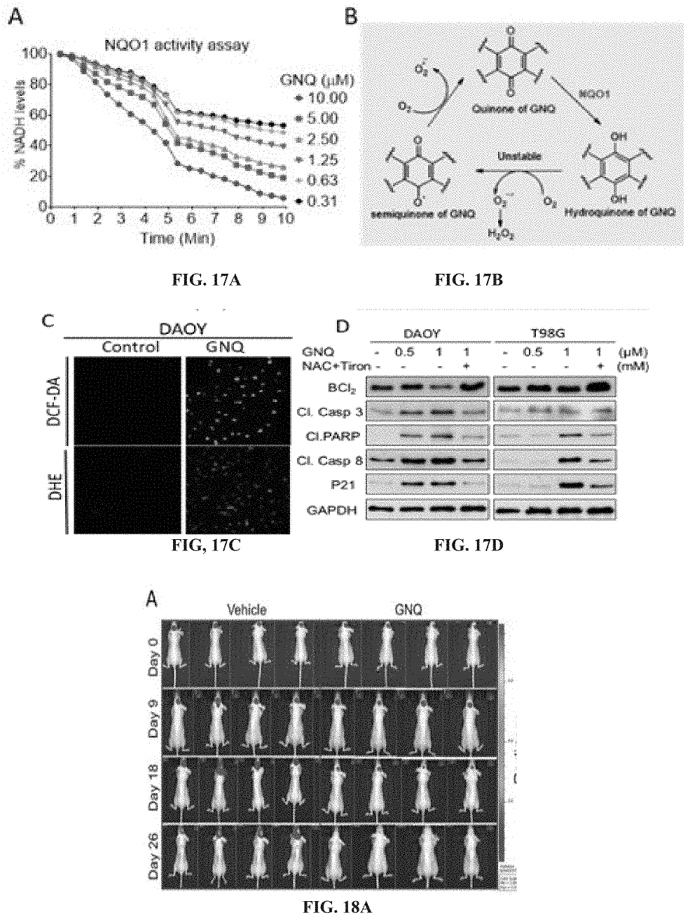

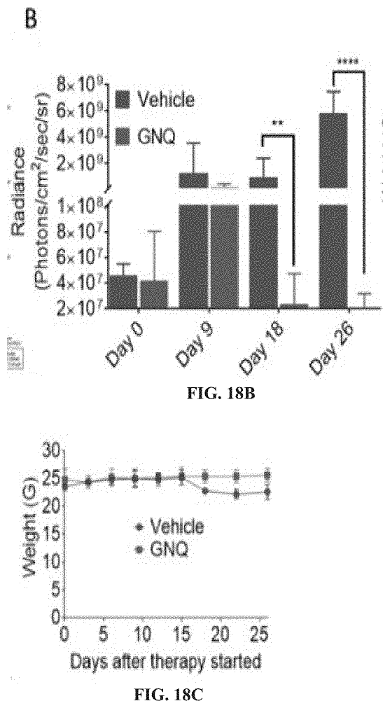

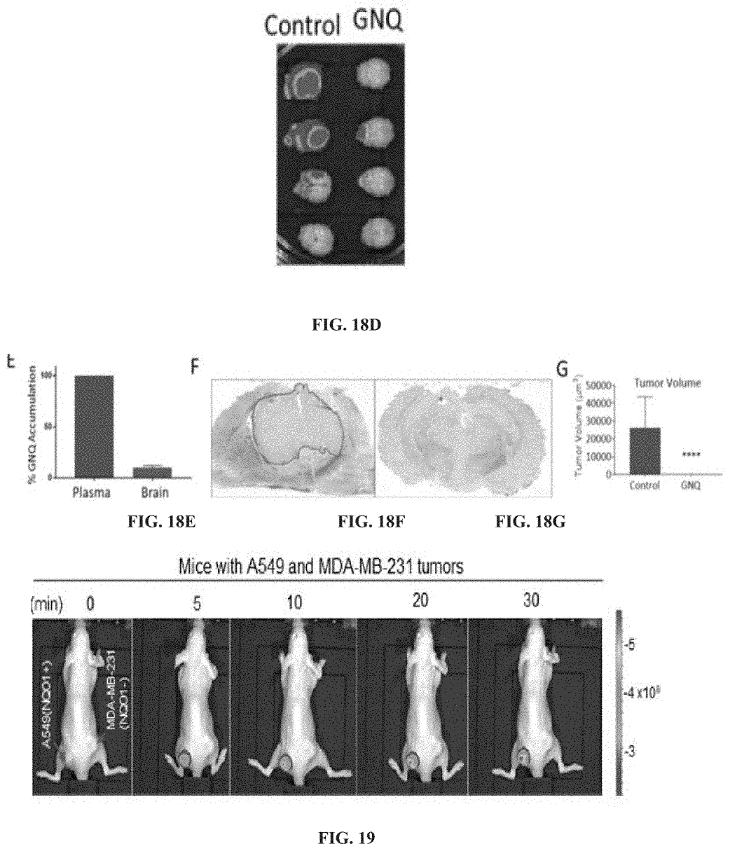

FIGS. 17A to 17D show that GNQ acts as an NQO1 substrate and generates Reactive oxygen species (ROS). As a substrate of NQO1 GNQ utilized greater than 1 equiv. of NADH throughout the assay, demonstrating the ability of this quinone to participate in redox cycling. (FIG. 17A). The generation of ROS by GNQ was assessed by using fluorescent probes DCFDA and DHE (FIG. 17C). GNQ acts as NQO1 substrate in vitro. (FIG. 17B) Reduction pathways and subsequent reactions of GNQ. (FIG. 17C) Intracellular ROS levels were measured using DCFDA and DHE. (FIG. 17D) Anti-oxidants reversed the GNQ induced apoptosis. Antitumor efficacy of GNQ against intracranial DAOY medulloblastoma: The BBB permeability of GNQ was examined by pharmacokinetic analysis after IV injections in normal mice. GNQ was found to accumulate in the brain up to 11% of its plasma levels (FIG. 18E). Intracranial DAOY tumors were set up in nude mice and the tumor growth was assessed by quantitative bioluminescence. GNQ triggered a complete loss of bioluminescence indicating tumor elimination (FIG. 18A). FIG. 18A, 18B show complete suppression of intracranial DAOY medulloblastoma growth by GNQ. GNQ was given IP at 25 mg/kg/d, 5 d/wk for about 4 weeks. (FIG. 18C) No changes in body weight as a surrogate marker for toxicity were noted. (FIG. 18D) Ex-vivo bioluminescence from the isolated brains (FIG. 18E) Accumulation of GNQ in the brain when compared to plasma. (FIG. 18F) H&E staining of mouse brain sections showing the elimination of GBM by GNQ. (FIG. 18G) Mean tumor volume radiance in the GNQ-treated group was compared with that in the control. Different substitutions (halogens, aryl, alkyl, and alkoxy groups) can be made to GNQ to append alkyl side chains to impart hydrophobicity to prepare BBB penetrating analogs.

Engineering of NQO1 turn-on near-infrared (NIR) fluorophores and theranostic drugs thereof for pediatric brain cancer therapy and multimodality imaging. There is an imperative need for the development of cancer-specific imaging and therapeutic multifunctional or theranostic agents that can allow both imaging and drug delivery. Imaging in the near-infrared (NIR) range of 650-900 nm has been widely used in clinical procedures, because of deep tissue penetration, low autofluorescence, and reduced photon scattering. The inventors developed enzyme-activatable, cell-permeable, non-toxic, and biocompatible ‘turn-on’ NIR fluorescent probes (NIR-ASM, NQ-DCP) that provide accurate detection and visualization of endogenous NQO1 activity both in vitro and an in vivo preclinical model of lung and brain cancers. These fluorophores remain non-fluorescent until cleavage by NQO1 catalysis. In this study, subcutaneous tumors of A549 lung cancer (NQO1-positive) and MDAMB-231 breast cancer (NQO1 negative) were developed in the same animal on opposite flanks.

FIG. 19 shows the infrared NQO1 fluorophore NIR-ASM (prepared by linking dicyanoisophorone with the NQO1 substrate quinone propionic acid) was injected at one mg/kg intravenously to the nude mice bearing NQO1-proficient A549 tumors on the left flank and NQO1-deficient MDAMB-231 tumors on the right flank. The animals were imaged in a IVIS imager at 500-640 nm. As can be seen, only the NQO1-positive A549 tumors showed signals within 5 min, and the NQO1-negative tumors were totally negative after 3 hours, demonstrating the specificity of the imaging reagents.

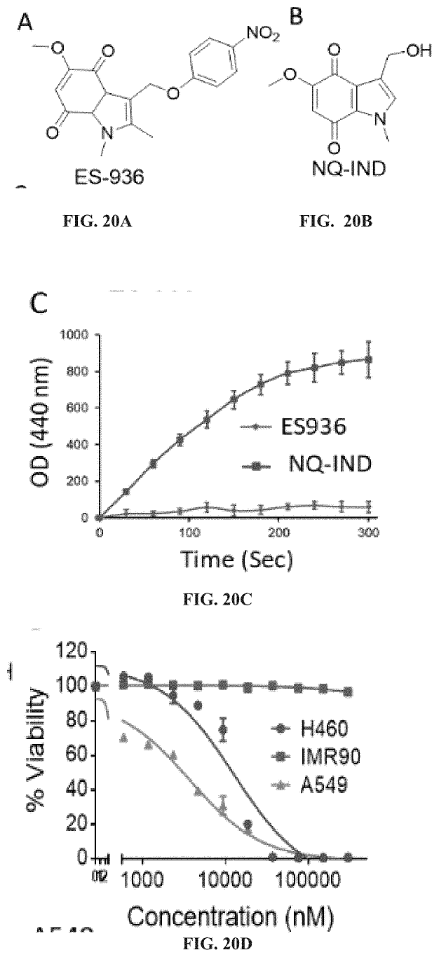

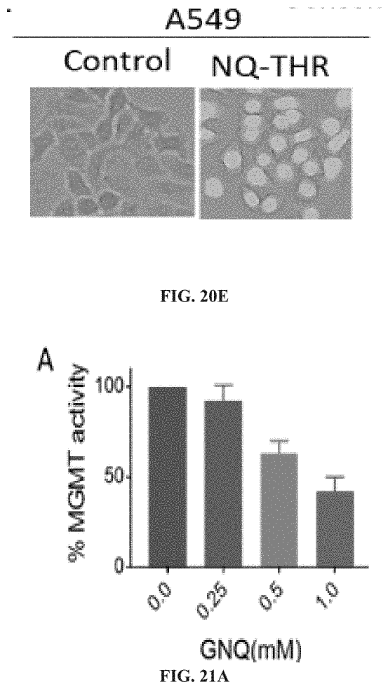

A tumor-targeted NIR small-molecule dye called NQ-THR, was made and specifically accumulated in cancer cells for tumor NIR imaging and simultaneously trigger tumoricidal oxidative stress by acting as an NQO1-bioactivatable substrate. This MR fluorophore provides sufficient specificity and imaging guidance to promote tumor-specific cell death, which can be used for the cancer treatment as well. NQ-IND, the precursor of NQ-THR is an NQO1 activated prodrug itself, which undergoes redox-cycling to produce ROS in brain tumor cells. The functional groups on NQ-IND are amenable to designing NQO1-activated imaging and therapeutic compounds. ES936 (FIG. 20A) is a highly specific, potent, mechanism-based NQO1 inhibitor (55). Inspired by the structure of ES936, the inventors prepared a compound NQ-IND (FIG. 20B) by removing the inhibitory nitroaryl group. NQ-IND was tested for its activity against NQO1 inhibition and cell viability. As shown in FIG. 20C, NQ-IND did not affect the activity of NQO1 while ES936 completely inhibited the NQO1 activity. However, NQIND was shown to inhibit cancer cell proliferation selectively with no effect on normal cells (FIG. 20D) and generate ROS as determined by DCF-DA fluorescence (FIG. 20E). These results indicate that NQ-IND acts as an efficient NQO1 substrate. The quinone NQ-IND can be covalently attached to the nitrogen mustard to prepare NQO1 activated prodrugs (NQ-IND NM). On the other hand, NQ-IND attached with the NIR fluorescent dyes can be used to generate a theranostic fluorescent probe NQ-THR with both imaging and therapeutic capabilities.

Next, the inventors sought to exploit the redox-induced DNA repair defects (oxidative inactivation of MGMT, PARP activation due to DNA-breaks created by H₂O₂) induced by NQO1 bioactivators for rational combination chemotherapy in childhood brain cancers.

The redox stress induced by NQO1 bioactivators is well known to generate peroxides, which in turn triggers DNA strand breaks and activation of PARP1. MGMT is a DNA repair protein and the single most important drug resistance determinant in brain tumors because it removes the methyl or chloroethyl groups introduced by anti-glioma alkylating agents at the 06 position of guanine. MGMT has an active-site cysteine to which the 06 alkyl groups from guanine are transferred in a stoichiometric self-inactivating reaction. The alkylated MGMT undergoes ubiquitination-dependent proteolysis. Such an MGMT-depleted state enables higher levels of alkylation damage by temozolomide (TMZ). The inventors have shown that MGMT is a redox-sensitive protein and it is inactivated in a prooxidant cell.

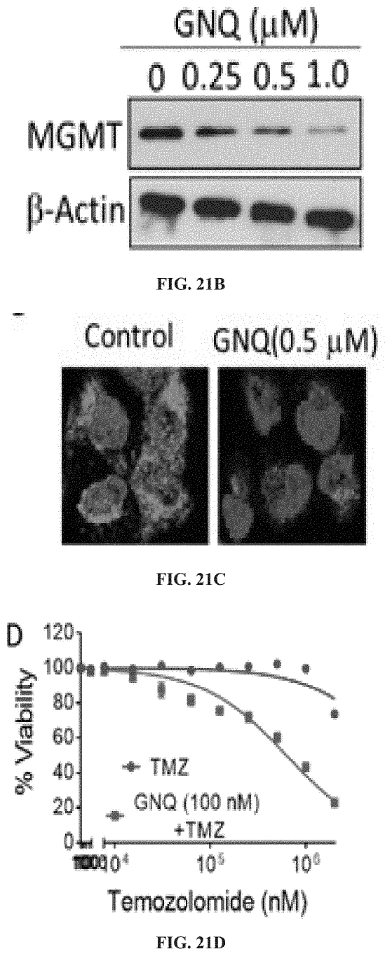

FIGS. 21A to 21D show that MGMT was remarkably inhibited by GNQ and DAOY cells were sensitized to TMZ. To further validate the MGMT deficiency, tumor cells exposed to GNQ can be treated with BCNU and the interstrand DNA crosslinking induced by BCNU, which can be quantitated by ethidium bromide fluorescence assay. Thus, it is possible to take advantage of these altered DNA repair pathways to combine GNQ with TMZ and PARP inhibitors and perform antitumor efficacy studies in orthotopic xenografts of pediatric brain cancers. Thus, NQO1 bioactivatable compounds exhibit significant anticancer efficacy by themselves and can also potentiate the activity of alkylating agents due to the simultaneous MGMT induced-deficiency.

FIG. 21A to 21D show the inhibition of MGMT activity (FIG. 21A) and its degradation (FIG. 21B) induced by GNQ in DAOY cells. (FIG. 21C) Immunofluorescence shows the decreased MGMT protein levels in GNQ treated cells. (FIG. 21D) GNQ potentiates the cytotoxicity of TMZ.

Statistical analyses. All experiments are performed at least three times independent of each other. The differences among control and treatment groups are tested using unpaired, two-tailed t-tests for comparison of two means or ANOVA for comparison of three or more groups. The sample numbers are adjusted, if necessary, to maintain a power of 0.8 to detect a 40% difference between the groups. For the number of animals, sample size calculations are carried out based on power analysis.

Numerous non-cancer diseases involve excessive or hyperproliferative cell growth, termed hyperplasia. As used herein, the terms “proliferative disorder”, “hyperproliferative disorder,” and “cell proliferation disorder” are used interchangeably to mean a disease or medical condition involving pathological growth of cells. Such disorders include cancer.

Non-cancerous proliferative disorders include smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis, desmoid tumors and the like.

Suitable pharmaceutically acceptable carriers may contain inert ingredients which do not inhibit the biological activity of the disclosed disalts. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, Mack Publishing Company, Easton, Pa. Formulation of the compound to be administered will vary according to the route of administration selected (e.g., solution, emulsion, capsule). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrins) are known in the art (Baker, et al., “Controlled Release of Biological. Active Agents”, John Wiley and Sons, 1986).

The compounds of the invention are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds of the invention can also be administered orally (e.g., dietary), topically, by inhalation (e.g., intrabronchial, intranasal, oral inhalation or intranasal drops), or rectally, depending on the type of cancer to be treated. Oral and parenteral administrations are preferred modes of administration.

As used in accordance with this invention, the term providing an effective amount means either directly administering such a compound of this invention, or administering a prodrug, derivative, or analog which will form an effective amount of the compound of this invention within the body.

Many new drugs are now available to be used by oncologists in treating patients with cancer. Often, tumors are more responsive to treatment when anti-cancer drugs are administered in combination to the patient than when the same drugs are administered individually and sequentially. One advantage of this approach is that the anti-cancer agents often act synergistically because the tumors cells are attacked simultaneously with agents having multiple modes of action. Thus, it is often possible to achieve more rapid reductions in tumor size by administering these drugs in combination. Another advantage of combination chemotherapy is that tumors are more likely to be eradicated completely and are less likely to develop resistance to the anti-cancer drugs being used to treat the patient.

Optionally, a compound of the invention, or a tautomer, pharmaceutically acceptable salt, solvate, clathrate, or a prodrug thereof, can be co-administered to treat a patient with a proliferative disorder such as cancer, or to prevent the reoccurrence of a proliferative disorder such as cancer, with other anti-cancer agents such as Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazornycin; edatrexate; eflornithine hydrochloride; elsamitrucin; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; viriorelbine tartrate; vinrosidine sulfate; vinzolidine sulfate; vorozole; zeniplatin; zinostatin; zorubicin hydrochloride.

Other drugs that can be used in combination with the compounds of the invention to treat a patient with a proliferative disorder such as cancer, or to prevent the reoccurrence of a proliferative disorder such as cancer, include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithineaelemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O⁶-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2, proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; rarnosetran; ras farnesyl protein transferase inhibitors; ras inhibitors; ras-GAP inhibitor; retelliptine demethylated; rhenium Re 186 etidronate; rhizoxin; ribozymes; Rh retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

It is understood that the effective dosage of the active compounds of this invention may vary depending upon the particular compound utilized, the mode of administration, the condition, and severity thereof, of the condition being treated, as well as the various physical factors related to the individual being treated. As used in accordance with invention, satisfactory results may be obtained when the compounds of this invention are administered to the individual in need at a daily dosage of from about 0.001 mg to about 100 mg per kilogram of body weight, preferably administered in divided doses two to six times per day, or in a sustained release form. For most large mammals, the total daily dosage is from about 1.5 mg to about 1000 mg. It is preferred that the administration of one or more of the compounds herein begin at a low dose and be increased until the desired effects are achieved.

Such doses may be administered in any manner useful in directing the active compounds herein to the recipient's bloodstream, including orally, via implants, and parenterally (including intravenous, intraperitoneal and subcutaneous injections). Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations herein may utilize standard delay or time release formulations to alter the absorption of the active compound(s).

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

It is contemplated that any embodiment discussed in this specification can be implemented with respect to any method, kit, reagent, or composition of the invention, and vice versa. Furthermore, compositions of the invention can be used to achieve methods of the invention. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A pharmaceutical composition comprising at least one of: cupric diethyldithiocarbamate (Cu-DDC), a DDC pro-drug, KSS-72, or 2,4-Dimethylene glutaric acid (DMG) in a pharmaceutically acceptable carrier for use in the treatment of tumor cells wherein the Cu-DDC, DDC pro-drug, KSS-72, or DMG directly or indirectly inhibit O⁶-methylguanine DNA methyltransferase in the tumor cells, wherein the amount is effective to potentiate an anti-tumor activity of one or more alkylating agents, platinum drugs, or antimetabolites, wherein tumor cells are triggered into programmed cell death.

2. The pharmaceutical composition of claim 1, wherein the Cu-DDC, DDC pro-drug, KSS-72, or DMG further comprise an albumin encapsulation, a liposomal encapsulation, or both.

3. The pharmaceutical composition of claim 1, wherein the DDC pro-drug is administered in combination with a metal chelate that includes an ion selected from the group consisting of arsenic, bismuth, cobalt, copper, chromium, gallium, gold iron, manganese, nickel, silver, titanium, vanadium, selenium and zinc.

4. The pharmaceutical composition of claim 1, further comprises a boronic acid hybrid, an ethacynic acid linked DDC, an N-acetyl lysine-linked DDC, or a gamma-glutamyl p-amido benzyl DDC.

5. The pharmaceutical composition of claim 1, wherein the DMG is provided in an amount sufficient to inhibit alpha keto glutarate (alpha KG) dehydrogenase to increase oxidative stress that inhibits O⁶-methylguanine DNA methyltransferase and triggers programmed cell death.

6. The pharmaceutical composition of claim 1, wherein the pharmaceutical composition is administered in a dosage of between 10, 20, 30 40, 50, 50, 60, 70, 75, 80, 90, 100, 200, 250, 300, 400, 500, 600, 700, 750, 800, 900 or 1000 mg of the Cu-DDC, DDC pro-drug, KSS-72, or DMG per day of body weight.

7. The pharmaceutical composition of claim 1, wherein the tumor cells are human brain tumor cells and the composition is formulated for intravenous or in situ administration.

8. The pharmaceutical composition of claim 1, wherein the tumor cells are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

9. The pharmaceutical composition of claim 1, wherein the one or more alkylating agents, platinum drugs, or antimetabolites are selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydro-xy-3,5-dimethoxyphenyl]-5,5a,6,8,8a,-9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2-,1-c]quinolin-7-one, and dimesna.

10. The pharmaceutical composition of claim 1, wherein the Cu-DDC, DDC pro-drug, KSS-72, or DMG composition is provided in an amount that inhibits O⁶-methylguanine DNA methyltransferase (MGMT) in situ.

11. The pharmaceutical composition of claim 1, further comprising temozolomide.

12. The pharmaceutical composition of claim 1, further comprising an activatable Cu-DDC, KSS-72, or DMG, wherein the Cu-DDC, KSS-72, or DMG are activatable in vivo with near infrared radiation, or the activatable Cu-DDC, DDC pro-drug, KSS-72, or DMG are conjugated with glutathione.

13. A pharmaceutical composition comprising an effective amount of an NAD(P)H Quinone Dehydrogenase 1 (NQO1) substrate in a pharmaceutically acceptable carrier that generates reactive oxygen species (ROS) in tumor cells, wherein processing of the NQO1 substrate in situ triggers programmed cell death in tumor cells by inhibiting O⁶-methylguanine DNA methyltransferase.

14. The pharmaceutical composition of claim 13, wherein the pharmaceutical composition is formulated for oral, parenterally, intravenous, or in situ administration.

15. The pharmaceutical composition of claim 13, wherein the pharmaceutical composition is administered in a dosage of between about 10, 20, 30 40, 50, 50, 60, 70, 75, 80, 90, 100, 200, 250, 300, 400, 500, 600, 700, 750, 800, 900 or 1000 mg per day of body weight.

16. The pharmaceutical composition of claim 13, wherein the tumor cells are human brain tumor cells.

17. The pharmaceutical composition of claim 13, wherein the tumor cells are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

18. The pharmaceutical composition of claim 13, further comprising a cytotoxic agents selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a,-9-hexohydrofuro(3′,′:6,7)naphtho(2,3- d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna.

19. The pharmaceutical composition of claim 13, wherein the Cu-DDC, KSS-72, or DMG composition is provided in an amount to inhibit O⁶-methylguanine DNA methyltransferase (MGMT) in situ.

20. The pharmaceutical composition of claim 13, further comprising temozolomide.

21. The pharmaceutical composition of claim 13, further comprising an activatable Cu-DDC, KSS-72, or DMG, wherein the Cu-DDC, KSS-72, or DMG are activatable in vivo with near infrared radiation, or the activatable Cu-DDC, KSS-72, or DMG is conjugated with glutathione.

22. A pharmaceutical composition comprising an effective amount of cupric diethyldithiocarbamate [Cu-DDC], KSS-72, or 2,4-Dimethylene glutaric acid (DMG) in a pharmaceutically acceptable carrier to inhibit O6-methylguanine DNA methyltransferase in tumor cells, wherein the Cu-DDC is formulated for oral, parenterally or in situ administration.

23. A chemotherapy composition comprising cupric diethyldithiocarbamate [Cu-DDC], KSS-72, or 2,4-Dimethylene glutaric acid (DMG), or activatable pro-GNQ, or pro-NQ-IND, disposed in a pharmaceutically acceptable carrier.

24. A chemotherapy composition comprising an effective amount of cupric diethyldithiocarbamate [Cu-DDC], Cu-DDC pro-drug, KSS-72, 2,4-Dimethylene glutaric acid (DMG), GNQ, pro-GNQ, NQ-IND, in an amount sufficient to inhibit an O⁶-methylguanine DNA methyltransferase disposed in a pharmaceutically acceptable carrier.

25. A theranostic agent comprising an NAD(P)H Quinone Dehydrogenase 1 (NQO1) substrate that generates reactive oxygen species (ROS) in tumor cells in cellulo and that comprises a detectable agent, wherein the detectable agent is used to diagnose the presence or absence of tumor cells, and when cleaved, the NQO1 substrate reduces or eliminates the tumor cells.

26. The theranostic agent of claim 25, wherein the detectable agent is a fluorophore.

27. The theranostic agent of claim 25, wherein the NQO1 substrate is GNQ or a functionalized derivative with substitutions selected from halogen, aryl, alkyl, or alkoxy group substitutions.

28. The theranostic agent of claim 25, wherein the NQO1 substrate is inducible.

29. The theranostic agent of claim 25, further comprising one or more one or more alkylating agents, platinum drugs, or antimetabolites are selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino [1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a,-9-hexohydrofuro(3′,′:6,7)naphtho(2,3- d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2-,1-c]quinolin-7-one, and dimesna.

30. The theranostic agent of claim 25, further comprising temozolomide.

31. The theranostic agent of claim 25, wherein the NQO1 substrate is: 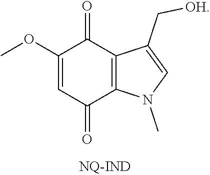 [SMILES: COC1=CC(=O)c2c(c(CO)cn2C)C1=O]
